(19) United States
(12) Patent Application Publication
(10) Pub. No.: US 2024/0042020 A1
(43) Pub. Date: Feb. 8, 2024

(54) ANTI-GARP-TGF-BETA1/PD-1 COMBINATION THERAPY

(71) Applicant: AbbVie Biotherapeutics Inc., North Chicago, IL (US)

(72) Inventors: Martha Blaney, El Granada, CA (US); Sarah Kongpachith, Pacifica, CA (US); James O’Brien, New York, NY (US); Josue Samayoa, San Mateo, CA (US)

(21) Appl. No.: 18/361,179

(22) Filed: Jul. 28, 2023

Related U.S. Application Data
(60) Provisional application No. 63/369,931, filed on Jul. 29, 2022.

(51) Int. Cl. A61K 39/395; C07K 16/28; C07K 16/22; A61K 31/282; A61K 31/519; A61K 31/337; A61K 31/7068; A61P 35/00

(52) U.S. Cl. CPC A61K 39/3955; C07K 16/2818; C07K 16/22; A61K 31/282; A61K 31/519; A61K 31/337; A61K 31/7068; A61P 35/00; C07K 2317/24; A61K 2039/505

(57) ABSTRACT

The present disclosure provides methods of treating cancer with the combination of an anti-TGF-β1/GARP complex antibody and an anti-PD-1 antibody.

### CROSS REFERENCE TO RELATED APPLICATIONS

This application claims the benefit of U.S. Provisional Application Ser. No. 63/369,931, filed Jul. 29, 2022, hereby incorporated by reference in its entirety.

### 1. SEQUENCE LISTING

The instant application contains a Sequence Listing which has been submitted electronically in XML format and is hereby incorporated by reference in its entirety. Said XML copy, created on Jul. 7, 2022, is named SeqList_350794-50100 and is 22,691 bytes in size.

### 2. TECHNICAL FIELD

The present application pertains to the use of antibodies capable of inhibiting activation of TGF-β1 in combination with checkpoint inhibitors for the treatment of cancer.

### 3. BACKGROUND

Glycoprotein-A repetitions predominant (GARP) binds to and regulates the availability of membrane-bound latent transforming growth factor beta-1 (TGF-β1) and modulates its activation. The GARP-TGF-β1 complex is expressed by several cell types including activated B cells, activated regulatory T lymphocytes, activated monocytes, and activated platelets. Upon release of active TGF-β1 from the GARP-TGF-β1 complex on activated regulatory T cells (Tregs), the TGF-β1 receptor on Tregs signals to enhance Treg immune-suppressive activity, whereas the receptor on tumor-infiltrating lymphocyte (TIL) serves to repress cytotoxic activity. Thus, TGF-β1 can act in an autocrine or paracrine fashion and can have different effects and functional outcomes on immune cells, ultimately leading to immunosuppression. Moreover, TGF-β1 has pleiotropic effects on other cell types harboring the receptor in tumor and peripheral tissues (de Streel and Lucas, 2021, Biochemical Pharmacology: 192:114697).

Monoclonal antibodies capable of interfering with the activation and release of mature TGF-β1 from GARP/TGF-β1 complexes were disclosed in WO 2015/015003 and WO 2016/125017. These antibodies have been shown to interfere with the immunosuppressive effects of Tregs in vitro and in vivo. WO 2018/206790 describes the humanization of ABBV-151 (livmoniplimab) a monoclonal antibody that specifically binds to the GARP-TGF-β1 complex, blocking release of active TGF-β1.

Preclinical data in mouse models using a surrogate antibody specific for the mouse GARP-TGF-β1 complex support the hypothesis that preventing release of TGF-β1 from the GARP-TGF-β1 complex results in loss of active TGF-β1 in the tumor and tissues where there may be cells that express the TGF-β1 receptor (de Streel, et al., 2020, Nature Communications, 11:4545).

To determine whether ABBV-151 when used in combination with antibodies targeting other immune checkpoint molecules can blunt the immunosuppressive effects of TGF-β1 and enable a more effective antitumor immune response, a phase 1 study was designed to determine the recommended Phase 2 dose (RP2D) of ABBV-151 administered as monotherapy and in combination with budigalimab (ABBV-181), an anti-PD-1 monoclonal antibody (see ClinicalTrials.gov Identifier NCT03821935).

### 4. SUMMARY

In embodiments, subjects having a cancer that evades host immunosurveillance at least partially through the expression and release of TGF-β1 are treated with the combination of an antibody that binds to a complex of human glycoprotein A repetitions predominant (hGARP) and TGF-β1, for example an Ab1, and an anti-PD-1 antibody. In some embodiments, the cancer is a solid tumor. In embodiments, the treatment of the cancer is a front line treatment, a second line treatment, or a second line plus treatment.

In embodiments, subjects having hepatocellular carcinoma are treated with the combination of an antibody that binds to a complex of human glycoprotein A repetitions predominant (hGARP) and TGF-β1, for example an Ab1, and an anti-PD-1 antibody. In embodiments, the treatment of hepatocellular carcinoma is a front line treatment, a second line treatment, or a second line plus treatment.

In embodiments, subjects having pancreatic adenocarcinoma are treated with the combination of an antibody that binds to a complex of human glycoprotein A repetitions predominant (hGARP) and TGF-β1, for example an Ab1, and an anti-PD-1 antibody.

In embodiments, subjects having urothelial cancer are treated with the combination of an antibody that binds to a complex of human glycoprotein A repetitions predominant (hGARP) and TGF-β1, for example an Ab1, and an anti-PD-1 antibody.

In embodiments, subjects having muscle invasive urothelial cancer are treated with the combination of an antibody that binds to a complex of human glycoprotein A repetitions predominant (hGARP) and TGF-β1, for example an Ab1, and an anti-PD-1 antibody.

In embodiments, subjects having head and neck squamous cell carcinoma are treated with the combination of an antibody that binds to a complex of human glycoprotein A repetitions predominant (hGARP) and TGF-β1, for example an Ab1, and an anti-PD-1 antibody.

In embodiments, subjects having microsatellite stable colorectal cancer are treated with the combination of an antibody that binds to a complex of human glycoprotein A repetitions predominant (hGARP) and TGF-β1, for example an Ab1, and an anti-PD-1 antibody. In embodiments, the microsatellite stable colorectal cancer is unselected. In other embodiments, the microsatellite stable colorectal cancer is the CMS4 subtype.

In embodiments, subjects having non-small cell lung cancer (NSCLC) are treated with the combination of an antibody that binds to a complex of human glycoprotein A repetitions predominant (hGARP) and TGF-β1, for example an Ab1, and an anti-PD-1 antibody. In embodiments, the treatment of NSCLC is a front-line treatment using a combination of Ab1, an anti-PD-1 antibody and chemotherapy. In embodiments, the chemotherapy is a platinum doublet regimen that uses carboplatin plus pemetrexed. In other embodiments, the combination of Ab1 and an anti-PD-1 antibody is used to treat relapsed/refractory NSCLC with or without liver metastasis.

In embodiments, subjects having ovarian granulosa cell tumors are treated with the combination of an antibody that binds to a complex of human glycoprotein A repetitions predominant (hGARP) and TGF-β1, for example, Ab1, and an anti-PD-1 antibody.

### 5. BRIEF DESCRIPTION OF THE DRAWINGS

FIG. 1. Study schema and enrollment status for the ongoing clinical trial NCT03821935.

FIGS. 2A and 2B. Dose escalation results for ABBV-151 monotherapy and ABBV-151+budigalimab combination therapy. Monotherapy: No responders. Combination cohorts: 4 confirmed responders and 1 unconfirmed responder; 4 with durable stable disease for ≥6 months. Indications include gastroesophageal junction adenocarcinoma (GEJ adenocarcinoma), colorectal cancer (CRC), ovarian cancer, alveolar sarcoma, and urothelial carcinoma.

FIG. 3. Dose expansion results for the PD-1 relapsed/refractory (R/R) urothelial carcinoma cohort: 5 confirmed responders and 1 unconfirmed responder; 5 with a best response of stable disease.

FIG. 4. Dose expansion results for the PD-1 naïve hepatocellular carcinoma cohort: 5 confirmed PRs (includes 1 PR per iRECIST and 1 that requires data cleaning); 3 with a best response of SD.

FIG. 5. Dose expansion results for the PD-1 naïve pancreatic adenocarcinoma cohort: 1 unconfirmed PR (new lesion at second scan); 6 with a best response of SD.

FIG. 6. Dose expansion results for the PD-1 naïve microsatellite stable colorectal cancer cohort: 1 confirmed PR (site entered overall response of SD but PR per lesion measurements): 7 with a best response of SD.

FIG. 7. Dose expansion results for 4 ovarian granuloma cell tumor patients: 3 out of 4 patients with confirmed PR.

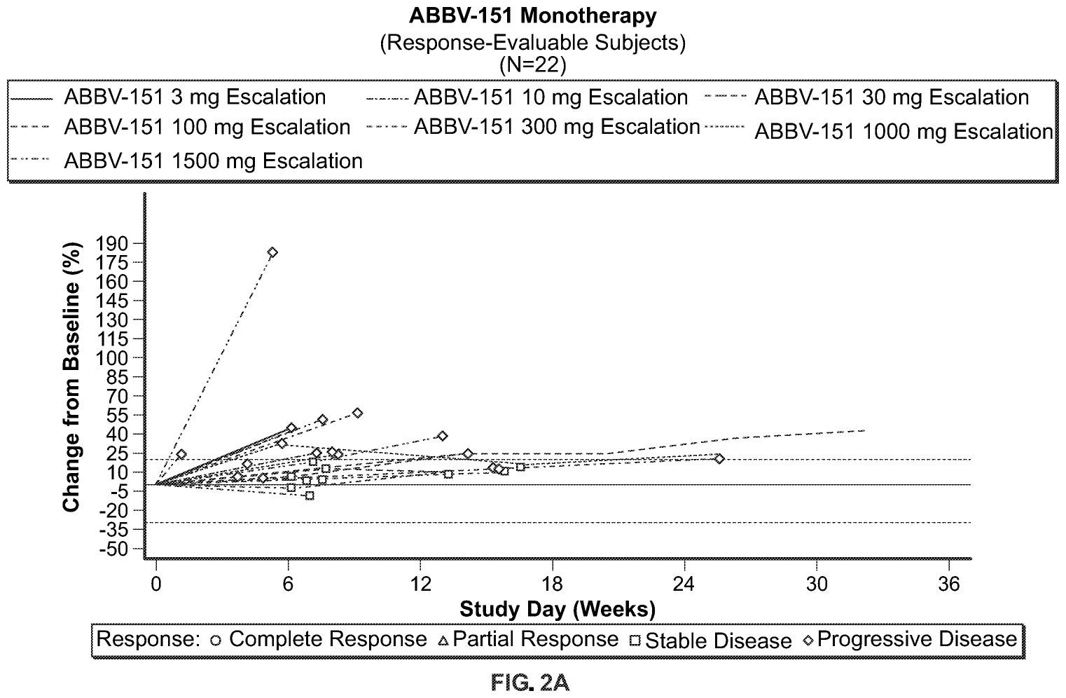

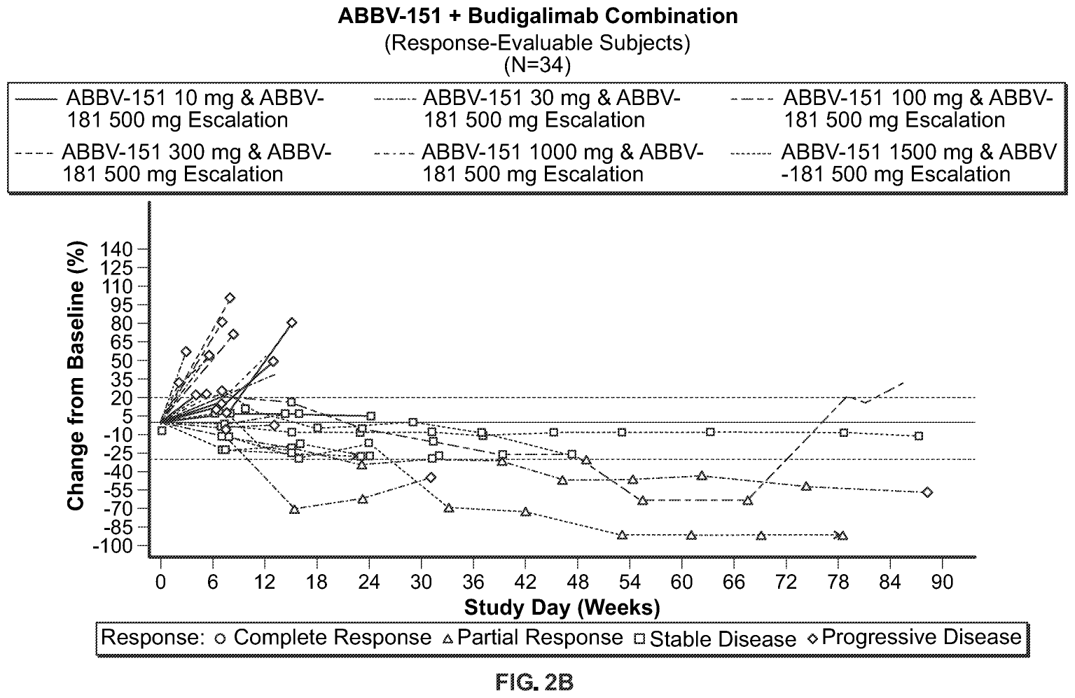

FIGS. 2A, 2B, 3, 4, 5, and 6: Shown are subjects with at least 1 post baseline disease assessment.

### 6. DETAILED DESCRIPTION

### 6.1. Antibodies

In one embodiment, provided is a method of treating cancer, the method comprising administering to a patient in need thereof (1) an antibody that binds to a complex of human glycoprotein A repetitions predominant (hGARP) and TGF-β1 and (2) an anti-PD-1 antibody.

In certain embodiments, the antibody that binds to the complex of hGARP and TGF-β1 is Ab1. Ab1, as used herein, refers to antibodies having the CDR sequences shown in Table 1. In embodiments, Ab1 is a human immunoglobulin G4 (IgG4; S228P)/k monoclonal antibody (mAb) that specifically binds to the GARP-TGF-β1 complex, blocking release of active TGF-β1. In embodiments, Ab1 comprises heavy chain variable regions (VH) of SEQ ID NO:7 and light chain variable regions (VL) of SEQ ID NO:8. In embodiments, Ab1 comprises heavy chains (HC) of SEQ ID NO:9 and light chains (LC) of SEQ ID NO:10. See Table 1 for amino acid sequences for the CDRs, variable and full-length sequences of Ab1. In an embodiment, the heavy chain sequences of Ab1 comprise the full-length heavy chain SEQ ID NO: 9 with an additional terminal lysine (K) residue (SEQ ID NO: 22).

In some embodiments, Ab1 is ABBV-151. ABBV-151, as used herein, refers to an antibody comprising heavy chains (HC) of SEQ ID NO:9 and light chains (LC) of SEQ ID NO:10.

In embodiments, Ab1 is livmoniplimab. Livmoniplimab, as used herein, refers to antibodies comprising heavy chains (HC) of SEQ ID NO:9 and light chains (LC) of SEQ ID NO:10 and products containing such antibodies wherein the antibodies or products have a name comprising the core name livmoniplimab with or without an FDA-designated suffix.

TABLE 1
Ab1, ABBV-151 and livmoniplimab sequences.

| SEQ ID NO | Amino acid sequence (N→C) |
|---|---|
| VH CDR1 | SYYID |
| SEQ ID NO: 1 | |
| VH CDR2 | RIDPEDAGTKYAQKFQG |
| SEQ ID NO: 2 | |
| VH CDR3 | YEWETVVVGDLMYEYEY |
| SEQ ID NO: 3 | |
| VL CDR1 | QASQSISSYLA |
| SEQ ID NO: 4 | |
| VL CDR2 | GASRLKT |
| SEQ ID NO: 5 | |
| VL CDR3 | QQYASVPVT |
| SEQ ID NO: 6 | |
| VH | QVQLVQPGAEVRKPGASVKVSCKASGYRFTSYYIDWVRQAPGQGLEWMGRIDPED |
| SEQ ID NO: 7 | AGTKYAQKFQGRVTMTADTSTSTVYVELSSLRSEDTAVYYCARYEWETVVVGDLM |
| | YEYEYWGQGTLVTVSS |
| VL | DIQMTQSPSSLSASVGDRVTITCQASQSISSYLAWYQQKPGQAPKILIYGASRLK |
| SEQ ID NO: 8 | TGVPSRESGSGSGTSFTLTISSLEPEDAATYYCQQYASVPVTFGQGTKVEIK |
| HC | QVQLVQPGAEVRKPGASVKVSCKASGYRFTSYYIDWVRQAPGOGLEWMGRIDPED |
| SEQ ID NO: 9 | AGTKYAQKFQGRVTMTADTSTSTVYVELSSLRSEDTAVYYCARYEWETVVVGDLM |
| | YEYEYWGQGTLVTVSSASTKGPSVFPLAPCSRSTSESTAALGCLVKDYFPEPVTV |
| | SWNSGALTSGVHTFPAVLOSSGLYSLSSVVTVPSSSLGTKTYTCNVDHKPSNTKV |
| | DKRVESKYGPPCPPCPAPEFLGGPSVFLFPPKPKDTLMISRTPEVTCVVVDVSQE |
| | DPEVQFNWYVDGVEVHNAKTKPREEQFNSTYRVVSVLTVLHODWLNGKEYKCKVS |
| | NKGLPSSIEKTISKAKGQPREPQVYTLPPSQEEMTKNQVSLTCLVKGFYPSDIAV |
| | EWESNGQPENNYKTTPPVLDSDGSFFLYSRLTVDKSRWQEGNVESCSVMHEALHN |
| | HYTQKSLSLSLG |
| LC | DIQMTQSPSSLSASVGDRVTITCQASQSISSYLAWYQQKPGQAPKILIYGASRLK |
| SEQ ID NO: | TGVPSRFSGSGSGTSFTLTISSLEPEDAATYYCQQYASVPVTFGQGTKVEIKRTV |
| 10 | AAPSVEIFPPSDEQLKSGTASVVCLLNNFYPREAKVOWKVDNALOSGNSQESVTE |
| | QDSKDSTYSLSSTLTLSKADYEKHKVYACEVTHQGLSSPVTKSENRGEC |

CDRs are underlined.
Constant regions are shown in italics.

The anti-PD-1 antibody may be an antibody that binds to PD-1. The anti-PD-1 antibody may be an antibody that binds to PD-L1. Antibodies that bind PD-1 may disrupt the binding of PD-1 to PD-L1 and PD-L2, and antibodies that bind PD-L1 may disrupt the binding of PD-1 to PD-L1.

In certain embodiments, the antibody that binds PD-1 is ABBV-181. ABBV-181, as used herein, refers to an antibody having the CDR sequences shown in Table 2. In embodiments, ABBV-181 as used herein refers to an antibody having a heavy chain of SEQ ID NO:19 or SEQ ID NO:21 and a light chain of SEQ ID NO:20. See Table 2 for amino acid sequences for the CDRs, variable and heavy chain sequences of ABBV-181.

In embodiments, ABBV-181 is budigalimab. Budigalimab, as used herein, refers to antibodies comprising heavy chains (HC) of SEQ ID NO:19 or SEQ ID NO:21 and light chains of SEQ ID NO:20 and products containing such antibodies wherein the antibodies or products have a name comprising the core name budigalimab with or without an FDA-designated suffix.

TABLE 2
ABBV-181 and budigalimab sequences.

| SEQ ID NO | Amino acid sequence (N→C) |
|---|---|
| VH CDR1 | GYTFTHYGMN |
| SEQ ID NO: 11 | |
| VH CDR2 | WVNTYTGEPTYADDEKG |
| SEQ ID NO: 12 | |
| VH CDR3 | EGEGLGFGD |
| SEQ ID NO: 13 | |
| VL CDR1 | RSSQSIVHSHGDTYLE |
| SEQ ID NO: 14 | |
| VL CDR2 | KVSNRFS |
| SEQ ID NO: 15 | |
| VL CDR3 | FOGSHIPVT |
| SEQ ID NO: 16 | |
| VH | EIQLVQSGAEVKKPGSSVKVSCKASGYTFTHYGMNWVRQAPGQGLEWVGWVNTY |
| SEQ ID NO: 17 | TGEPTYADDFKGRLTFTLDTSTSTAYMELSSLRSEDTAVYYCTREGEGLGFGDW |
| | GQGTTVTVSS |
| VL | DVVMTQSPLSLPVTPGEPASISCRSSQSIVHSHGDTYLEWYLQKPGOSPQLLIY |
| SEQ ID NO: 18 | KVSNRFSGVPDRFSGSGSGTDFTLKISRVEAEDVGVYYCFQGSHIPVTFGQGTK |
| | LEIKR |
| HC #1 | EIQLVQSGAEVKKPGSSVKVSCKASGYTFTHYGMNWVRQAPGQGLEWVGWVNTY |
| SEQ ID NO: 19 | TGEPTYADDFKGRLTFTLDTSTSTAYMELSSLRSEDTAVYYCTREGEGLGFGDW |
| | GQGTTVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKV |
| | EPKSCDKTHTCPPCPAPEAAGGPSVFLFPPKPKDTLMISRTPEVTCVVVDVSHE |
| | DPEVKENWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPSDI |
| | AVEWESNGQPENNYKTTPPVLDSDGSFFLYSKLTVDKSRWQQGNVESCSVMHEA |
| | LHNHYTQKSLSLSPGK |
| LC | DVVMTQSPLSLPVTPGEPASISCRSSQSIVHSHGDTYLEWYLQKPGOSPQLLIY |
| SEQ ID NO: 20 | KVSNRFSGVPDRFSGSGSGTDFTLKISRVEAEDVGVYYCFQGSHIPVTFGQGTK |
| | LEIKRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSENR |
| | GEC |
| HC #2 | EIQLVQSGAEVKKPGSSVKVSCKASGYTFTHYGMNWVRQAPGQGLEWVGWVNTY |
| SEQ ID NO: 21 | TGEPTYADDFKGRLTFTLDTSTSTAYMELSSLRSEDTAVYYCTREGEGLGFGDW |
| | GQGTTVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKV |
| | EPKSCDKTHTCPPCPAPEAAGGPSVELFPPKPKDTLMISRTPEVTCVVVDVSHE |
| | DPEVKENWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPSDI |
| | AVEWESNGQPENNYKTTPPVLDSDGSFFLYSKLTVDKSRWQQGNVFSCSVMHEA |
| | LHNHYTQKSLSLSPG |

CDRs are underlined.
Constant regions are shown in italics.

In one embodiment, provided is a method of treating cancer, the method comprising administering to a patient in need thereof (1) an antibody that binds to a complex of hGARP and TGF-β1, e.g., Ab1 and (2) an anti-PD-1 antibody. In one embodiment, the anti-PD-1 antibody binds PD-1 and is selected from the group consisting of pembrolizumab, nivolumab, cemiplimab, dostarlimab, and budigalimab. In one embodiment the method of treating cancer comprises administration to a patient in need thereof (1) an antibody that binds to a complex of hGARP and TGF-β1, e.g., Ab1, and (2) an antibody that binds PD-L1 selected from the group consisting of atezolizumab, durvalumab, and avelumab.

### 6.2. Administration of the Antibody that Binds to a Complex of hGARP and TGF-β1 and an Anti-PD-1 Antibody

Provided herein are methods for treating patients with cancer with the combination of an antibody that binds to a complex of hGARP and TGF-β1 and an anti-PD-1 antibody. In some embodiments, the cancer is a solid tumor. Provided below are non-limiting examples of timing and dosages of the antibody that binds to a complex of hGARP and TGF-β1 appropriate for use in combination with anti-PD-1 antibodies, non-limiting examples of timing and dosages of the anti-PD-1 antibody appropriate for use in combination with an antibody that binds to a complex of hGARP and TGF-β1, and non-limiting examples of the administration of both types of antibodies.

6.2.1. Administration of the Antibody that Binds to a Complex of hGARP and TGF-β1

In some embodiments, the antibody that binds to a complex of hGARP and TGF-β1 is administered as an IV infusion once every week (Q1W), once every 2 weeks (Q2W), once every 3 weeks (Q3W), or once every 4 weeks (Q4W).

In some embodiments, the antibody that binds to a complex of hGARP and TGF-β1 is administered as a flat dose of 200 mg through 1500 mg Q2W. In some embodiments, the antibody that binds to a complex of hGARP and TGF-β1 is administered as a flat dose of 200, 250, 300, 400, 500, 750, 1000, or 1500 mg Q2W. In yet other embodiments the antibody that binds to a complex of hGARP and TGF-β1 is administered as a flat dose of 1500 mg Q2W.

In some embodiments, the antibody that binds to a complex of hGARP and TGF-β1 is administered as an IV infusion at a flat dose of from 200 mg through 1200 mg Q3W. In some embodiments, the antibody that binds to a complex of hGARP and TGF-β1 is administered as an IV infusion at a flat dose of 200, 400, 600, 800, or 1200 mg Q3W. In some embodiments, the antibody that binds to a complex of hGARP and TGF-β1 is administered as an IV infusion at a flat dose of 400 mg Q3W. In some embodiments, the antibody that binds to a complex of hGARP and TGF-β1 is administered as an IV infusion at a flat dose of 1200 mg Q3W.

In some embodiments, the antibody that binds to a complex of hGARP and TGF-β1 is administered as an IV infusion at a flat dose of 250 mg through 1600 mg Q4W. In some embodiments, the antibody that binds to a complex of hGARP and TGF-β1 is administered as an IV infusion at a flat dose of 250, 500, 550, 600, 750, 1000, or 1500 mg Q4W. In some embodiments, the antibody that binds to a complex of hGARP and TGF-β1 is administered as an IV infusion at a flat dose of 600 mg Q4W. In some embodiments the antibody that binds to a complex of hGARP and TGF-β1 is administered as an IV infusion at a flat dose of 1500 mg Q4W.

In embodiments, the antibody that binds to a complex of hGARP and TGF-β1 is administered in an amount sufficient and on a schedule sufficient to improve the therapeutic efficacy of the anti-PD-1 antibody. In some embodiments, the antibody that binds to a complex of hGARP and TGF-β1 is administered at a dose that achieves: (1) a concentration of 0.8 ug/mL at the tumor site which is minimally required to inhibit TGF-β1 signaling at the site of action, for example, the tumor site, and (2) the antibody that binds to a complex of hGARP and TGF-β1 at a dose of 0.319 ug/mL, that achieves the EC₉₅ for GARP/TGF-β1 target engagement in the tumor microenvironment for the majority of subjects.

In an embodiment, the antibody that binds to a complex of hGARP and TGF-β1 in this section (Section 6.2.1) is Ab1. In an embodiment, the antibody that binds to a complex of hGARP and TGF-β1 in this section (Section 6.2.1) is Ab1, wherein Ab1 comprises CDRH1, CDRH2 and CDRH3 of SEQ ID NOs: 1, 2 and 3, respectively, and CDRL1, CDRL2 and CDRL3 of SEQ ID Nos: 4, 5 and 6, respectively. In an embodiment, the antibody that binds to a complex of hGARP and TGF-β1 in this section (Section 6.2.1) is Ab1, wherein Ab1 comprises heavy chain variable regions of SEQ ID NO:7 and light chain variable regions of SEQ ID NO:8. In an embodiment, the antibody that binds to a complex of hGARP and TGF-β1 in this section (Section 6.2.1) is Ab1, wherein Ab1 comprises heavy chains of SEQ ID NO:9 and light chains of SEQ ID NO:10. In an embodiment, the antibody that binds to a complex of hGARP and TGF-β1 in this section (Section 6.2.1) is Ab1, wherein Ab1 comprises heavy chains of SEQ ID NO:22 and light chains of SEQ ID NO:10.

In an embodiment, the antibody that binds to a complex of hGARP and TGF-β1 in this section (Section 6.2.1) is Ab1, wherein Ab1 is ABBV-151, comprising heavy chains (HC) of SEQ ID NO:9 and light chains (LC) of SEQ ID NO:10.

In an embodiment, the antibody that binds to a complex of hGARP and TGF-β1 in this section (Section 6.2.1) is Ab1, wherein Ab1 is livmoniplimab, comprising heavy chains (HC) of SEQ ID NO:9 and light chains (LC) of SEQ ID NO:10.

6.2.2. Administration of the Anti-PD-1 Antibody

In some embodiments, the anti-PD-1 antibody is administered at a dose of 1-10 mg/kg once every 2 weeks (Q2W), once every three weeks (Q3W) or once every 4 weeks (Q4W). In some embodiments, the anti-PD-1 antibody is administered at a flat dose of 240 mg-1680 mg once every 2 weeks (Q2W), once every three weeks (Q3W), once every 4 weeks (Q4W) or once every 6 weeks (Q6W).

In some embodiments, the anti-PD-1 antibody is administered at a dose of 1, 3 or 10 mg/kg once every 2 weeks (Q2W), once every three weeks (Q3W) or once every 4 weeks (Q4W). In some embodiments, the anti-PD-1 antibody is administered at a flat dose of 240 mg once every 2 weeks (Q2W), 250 mg once every two weeks (Q2W), 840 mg once every 2 weeks (Q2W), 200 mg once every 3 weeks (Q3W), 360 mg once every 3 weeks (Q3W), 375 mg once every three weeks (Q3W), 1200 mg once every 3 weeks (Q3W), 480 mg once every 4 weeks (Q4W), 500 mg once every 4 weeks (Q4W), 1680 mg once every 4 weeks (Q4W), or 400 mg once every 6 weeks (Q6W). In some embodiments, the anti-PD-1 antibody is administered at a flat dose of 375 mg Q3W to subjects in combination cohorts only. In one embodiment, the first infusion is administered over 90 minutes. If a subject does not experience any infusion-related reactions during the first dose, the duration of infusion for the second dose may be shortened to 60 minutes and for subsequent doses may be shortened to 30 minutes.

6.2.2.1. Administration of ABBV-181

In some embodiments, the anti-PD-1 antibody is ABBV-181 and is administered at a dose of 1, 3 or 10 mg/kg once every 2 weeks (Q2W), once every three weeks (Q3W) or once every 4 weeks (Q4W). In some embodiments, the anti-PD-1 antibody is administered at a flat dose of 250 mg once every two weeks (Q2W), 375 mg once every three weeks (Q3W), or 500 mg once every 4 weeks (Q4W).

In some embodiments, the anti-PD-1 antibody is ABBV-181 and is administered at a dose of 1, 3, or 10 mg/kg once every 2 weeks (Q2W). In some embodiments, the anti-PD-1 antibody is ABBV-181 and is administered at a flat dose of 250, 375, or 500 mg once every 2 weeks (Q2W). In some embodiments, ABBV-181 is administered at a flat dose of 250 mg Q2W to subjects in combination cohorts only. In one embodiment, the first infusion is administered over 90 minutes. If a subject does not experience any infusion-related reactions during the first dose, the duration of infusion for the second dose may be shortened to 60 minutes and for subsequent doses may be shortened to 30 minutes.

In some embodiments, the anti-PD-1 antibody is ABBV-181 and is administered at a dose of 1, 3, or 10 mg/kg once every 3 weeks (Q3W). In some embodiments, the anti-PD-1 antibody is ABBV-181 and is administered at a flat dose of 250, 375, or 500 mg once every 3 weeks (Q3W). In some embodiments, ABBV-181 is administered at a flat dose of 375 mg Q3W to subjects in combination cohorts only. In one embodiment, the first infusion is administered over 90 minutes. If a subject does not experience any infusion-related reactions during the first dose, the duration of infusion for the second dose may be shortened to 60 minutes and for subsequent doses may be shortened to 30 minutes.

In some embodiments, ABBV-181 is administered at a dose of 1, 3 or 10 mg/kg or a flat dose of 250, 375, or 500 mg once every 4 weeks (Q4W). In some embodiments, ABBV-181 is administered at a flat dose of 500 mg Q4W to subjects in combination cohorts only. In one embodiment, the first infusion is administered over 90 minutes. If a subject does not experience any infusion-related reactions during the first dose, the duration of infusion for the second dose may be shortened to 60 minutes and for subsequent doses may be shortened to 30 minutes.

6.2.2.2. Administration of Budigalimab

In some embodiments, the anti-PD-1 antibody is budigalimab and is administered at a dose of 1, 3 or 10 mg/kg once every 2 weeks (Q2W), once every three weeks (Q3W) or once every 4 weeks (Q4W). In some embodiments, the anti-PD-1 antibody is administered at a flat dose of 250 mg once every two weeks (Q2W), 375 mg once every three weeks (Q3W), or 500 mg once every 4 weeks (Q4W).

In some embodiments, the anti-PD-1 antibody is budigalimab and is administered at a dose of 1, 3, or 10 mg/kg once every 2 weeks (Q2W). In some embodiments, the anti-PD-1 antibody is budigalimab and is administered at a flat dose of 250, 375, or 500 mg once every 2 weeks (Q2W). In some embodiments, budigalimab is administered at a flat dose of 250 mg Q2W to subjects in combination cohorts only. In one embodiment, the first infusion is administered over 90 minutes. If a subject does not experience any infusion-related reactions during the first dose, the duration of infusion for the second dose may be shortened to 60 minutes and for subsequent doses may be shortened to 30 minutes.

In some embodiments, the anti-PD-1 antibody is budigalimab and is administered at a dose of 1, 3 or 10 mg/kg, or a flat dose of 250, 375, or 500 mg once every 3 weeks (Q3W). In some embodiments, budigalimab is administered at a flat dose of 375 mg Q3W to subjects in combination cohorts only. In one embodiment, the first infusion is administered over 90 minutes. If a subject does not experience any infusion-related reactions during the first dose, the duration of infusion for the second dose may be shortened to 60 minutes and for subsequent doses may be shortened to 30 minutes.

In some embodiments, budigalimab is administered at a dose of 1, 3 10 mg/kg once every 4 weeks (Q4W). In some embodiments, budigalimab is administered at a flat dose of 250, 375, or 500 mg once every 4 weeks (Q4W). In some embodiments, budigalimab is administered at a flat dose of 500 mg Q4W to subjects in combination cohorts only. In one embodiment, the first infusion is administered over minutes. If a subject does not experience any infusion-related reactions during the first dose, the duration of infusion for the second dose may be shortened to 60 minutes and for subsequent doses may be shortened to 30 minutes.

6.2.2.3. Administration of Pembrolizumab, Nivolumab and Atezolizumab

In some embodiments, the anti-PD-1 antibody is pembrolizumab and is administered at a flat dose of 200 mg once every 3 weeks (Q3W). In some embodiments, the anti-PD-1 antibody is pembrolizumab and is administered at a flat dose of 400 mg once every 6 weeks (Q6W). Doses and frequencies of administration of pembrolizumab are known in the art, for example, as specified in the KEYTRUDA® Prescribing Information.

In some embodiments, the anti-PD-1 antibody is nivolumab and is administered at a flat dose of 240 mg once every 2 weeks (Q2W). In some embodiments, the anti-PD-1 antibody is nivolumab and is administered at 3 mg/kg once every 2 weeks (Q2W). In some embodiments, the anti-PD-1 antibody is nivolumab and is administered at a flat dose of 360 mg once every 3 weeks (Q3W). In some embodiments, the anti-PD-1 antibody is nivolumab and is administered at a flat dose of 480 mg once every 4 weeks (Q4W). Doses and frequencies of administration of nivolumab are known in the art, for example, as specified in the OPDIVO® Prescribing Information.

In some embodiments, the anti-PD-1 antibody binds PD-L1, is atezolizumab, and is administered at a flat dose of 840 mg once every 2 weeks (Q2W). In some embodiments, the anti-PD-1 antibody binds PD-L1, is atezolizumab, and is atezolizumab and is administered at a flat dose of 1200 mg once every 3 weeks (Q3W). In some embodiments, the anti-PD-1 antibody binds PD-L1, is atezolizumab, and is administered at a flat dose of 1680 mg once every 4 weeks (Q4W). Doses and frequencies of administration of atezolizumab are known in the art, for example, as specified in the TECENTRIQ® Prescribing Information.

6.2.3. Combination Regimens

The following combination regimens are provided as non-limiting examples, and omission of a particular combination of timing and dosages does not indicate that that combination has not been explicitly contemplated or is not within scope of the invention disclosed herein.

In one embodiment, (1) the antibody that binds to a complex of hGARP and TGF-β1 and (2) the anti-PD-1 antibody are administered simultaneously.

In one embodiment, (1) the antibody that binds to a complex of hGARP and TGF-β1 and (2) the anti-PD-1 antibody are administered consecutively. In certain embodiments, the antibody that binds to a complex of hGARP and TGF-β1 is administered first, followed by the administration of the anti-PD-1 antibody. In embodiments, up to 15 minutes, 30 minutes, 45 minutes, or 60 minutes elapse before the administration of the anti-PD-1 antibody.

In one embodiment, (1) the antibody that binds to a complex of hGARP and TGF-β1 and (2) the anti-PD-1 antibody are administered non-simultaneously within 4 weeks, 3 weeks, 2 weeks, 1 week, 2 days, or 1 day of one another.

In some embodiments, the antibody that binds to a complex of hGARP and TGF-β1 is administered at a flat dose of 200 mg through 1500 mg Q2W and the anti-PD-1 antibody is ABBV-181 or budigalimab and is administered at a flat dose of 500 mg (Q4W). In some embodiments, the antibody that binds to a complex of hGARP and TGF-β1 is administered at a flat dose of 200, 250, 300, 400, 500, 750, 1000, or 1500 mg Q2W and the anti-PD-1 antibody is ABBV-181 or budigalimab and is administered at a flat dose of 500 mg (Q4W). In some embodiments, the antibody that binds to a complex of hGARP and TGF-β1 is administered at a flat dose of 400 mg Q2W and the anti-PD-1 antibody is ABBV-181 or budigalimab and is administered at a flat dose of 500 mg (Q4W). In some embodiments, the antibody that binds to a complex of hGARP and TGF-β1 is administered at a flat dose of 600 mg Q2W and the anti-PD-1 antibody is ABBV-181 or budigalimab and is administered at a flat dose of 500 mg (Q4W). In some embodiments, the antibody that binds to a complex of hGARP and TGF-β1 is administered at a flat dose of 1500 mg Q2W and the anti-PD-1 antibody is ABBV-181 or budigalimab and is administered at a flat dose of 500 mg (Q4W).

In some embodiments, the antibody that binds to a complex of hGARP and TGF-β1 is administered at a flat dose of 200 mg through 1200 mg Q3W and the anti-PD-1 antibody is ABBV-181 or budigalimab and is administered at a flat dose of 375 mg (Q3W). In some embodiments, the antibody that binds to a complex of hGARP and TGF-β1 is administered at a flat dose of 200, 400, 600, 800, or 1200 mg Q3W and the anti-PD-1 antibody is ABBV-181 or budigalimab and is administered at a flat dose of 375 mg (Q3W). In some embodiments, the antibody that binds to a complex of hGARP and TGF-β1 is administered at a flat dose of 400 mg Q3W and the anti-PD-1 antibody is ABBV-181 or budigalimab and is administered at a flat dose of 375 mg (Q3W). In some embodiments, the antibody that binds to a complex of hGARP and TGF-β1 is administered at a flat dose of 600 mg Q3W and the anti-PD-1 antibody is ABBV-181 or budigalimab and is administered at a flat dose of 375 mg (Q3W). In some embodiments, the antibody that binds to a complex of hGARP and TGF-β1 is administered at a flat dose of 1200 mg Q3W and the anti-PD-1 antibody is ABBV-181 or budigalimab and is administered at a flat dose of 375 mg (Q3W).

In some embodiments, the antibody that binds to a complex of hGARP and TGF-β1 is administered at a flat dose of 250 mg through 1600 mg Q4W and the anti-PD-1 antibody is ABBV-181 or budigalimab and is administered at a flat dose of 500 mg (Q4W). In some embodiments, the antibody that binds to a complex of hGARP and TGF-β1 is administered at a flat dose of 250, 500, 550, 600, 750, 1000, 1500 mg, 1600 mg Q4W and the anti-PD-1 antibody is ABBV-181 or budigalimab and is administered at a flat dose of 500 mg (Q4W). In some embodiments, the antibody that binds to a complex of hGARP and TGF-β1 is administered at a flat dose of 600 mg Q4W and the anti-PD-1 antibody is ABBV-181 or budigalimab and is administered at a flat dose of 500 mg (Q4W). In some embodiments, the antibody that binds to a complex of hGARP and TGF-β1 is administered at a flat dose of 1500 mg Q4W and the anti-PD-1 antibody is ABBV-181 or budigalimab and is administered at a flat dose of 500 mg (Q4W).

In embodiments, the order of drug administration is the antibody that binds to a complex of hGARP and TGF-β1 first, followed by the anti-PD-1 antibody. In embodiments, following the completion of the antibody that binds to a complex of hGARP and TGF-β1 infusion, subjects wait up to 60 minutes before starting the anti-PD-1 antibody infusion.

In embodiments, administration of the combination of the antibody that binds to a complex of hGARP and TGF-β1 and the anti-PD-1 antibody is continued until either disease progression or unacceptable toxicity occurs. In embodiments, administration of the combination of the antibody that binds to a complex of hGARP and TGF-β1 and the anti-PD-1 antibody is continued for 4 months, 5 months, 6 months, 7 months, 8 months, 12 months, 18 months, 24 months, or longer.

Efficacy of the combination of the antibody that binds to a complex of hGARP and TGF-β1 and the anti-PD-1 antibody is assessed through various clinical endpoints. In embodiments, subjects treated with the combination of the antibody that binds to a complex of hGARP and TGF-β1 and the anti-PD-1 antibody, have an objective response rate (ORR) greater than the standard of care. In embodiments, subjects treated with the combination of the antibody that binds to a complex of hGARP and TGF-β1 and the anti-PD-1 antibody, have an objective response rate (ORR) greater than that observed with administration of the anti-PD-1 antibody alone. In embodiments, subjects treated with the combination of the antibody that binds to a complex of hGARP and TGF-β1 and the anti-PD-1 antibody, have an objective response rate (ORR) greater than or equal to 5%, greater than or equal to 10%, greater than or equal to 15%, greater than or equal to 20%, greater or equal to 25%, greater than or equal to 30%, greater or equal to 40%, greater than or equal to 50%, greater than or equal to 60%, or greater than or equal to 70%. In embodiments, efficacy of the treatment includes a median duration of response (DoR) of 4 months or more (e.g, of at least 4 months, at least 6 months, at least 8 months, and/or at least 10 months). Other efficacy endpoints include disease free survival (DFS), progression free survival (PFS), overall survival (OS), and an acceptable safety and tolerability profile.

In an embodiment, the antibody that binds to a complex of hGARP and TGF-β1 in this section (Section 6.2.3) is Ab1. In an embodiment, the antibody that binds to a complex of hGARP and TGF-β1 in this section (Section 6.2.3) is Ab1, wherein Ab1 comprises CDRH1, CDRH2 and CDRH3 of SEQ ID NOs: 1, 2 and 3, respectively, and CDRL1, CDRL2 and CDRL3 of SEQ ID Nos: 4, 5 and 6, respectively. In an embodiment, the antibody that binds to a complex of hGARP and TGF-β1 in this section (Section 6.2.3) is Ab1, wherein Ab1 comprises heavy chain variable regions of SEQ ID NO:7 and light chain variable regions of SEQ ID NO:8. In an embodiment, the antibody that binds to a complex of hGARP and TGF-β1 in this section (Section 6.2.3) is Ab1, wherein Ab1 comprises heavy chains of SEQ ID NO:9 and light chains of SEQ ID NO:10. In an embodiment, the antibody that binds to a complex of hGARP and TGF-β1 in this section (Section 6.2.3) is Ab1, wherein Ab1 comprises heavy chains of SEQ ID NO:22 and light chains of SEQ ID NO:10.

In an embodiment, the antibody that binds to a complex of hGARP and TGF-β1 in this section (Section 6.2.3) is Ab1, wherein Ab1 is ABBV-151, comprising heavy chains (HC) of SEQ ID NO:9 and light chains (LC) of SEQ ID NO:10.

In an embodiment, the antibody that binds to a complex of hGARP and TGF-β1 in this section (Section 6.2.3) is Ab1, wherein Ab1 is livmoniplimab, comprising heavy chains (HC) of SEQ ID NO:9 and light chains (LC) of SEQ ID NO:10.

In an embodiment, the anti-PD-1 antibody in this section (Section 5.2.3) is ABBV-181. In an embodiment, the anti-PD-1 antibody in this section (Section 6.2.3) is budigalimab. In an embodiment, the anti-PD-1 antibody in this section (Section 6.2.3) is nivolumab. In an embodiment, the anti-PD-1 antibody in this section (Section 6.2.3) is pembrolizumab. In an embodiment, the anti-PD-1 antibody in this section (Section 6.2.3) is atezolizumab.

### 6.3. Selection of Tumor Types

It has been proposed that there may be 3 basic cancer immune phenotypes: desert (characterized by the absence of immune infiltrate in tumor or surrounding stroma), excluded (in which immune cells are present in the stroma but are unable to access the tumor microenvironment), and inflamed (in which immune cells are present in the stroma and tumor microenvironment). Furthermore, the success of immunotherapies targeting T-cell co-stimulation, such as anti-PD-1 agents, may rely on both the content and location of T cell infiltrates (Chen D S, Mellman I. Nature. 2017; 541(7637):321-30).

Gene expression analysis was used to compare markers of immune infiltration and TGF-β1-related signaling in multiple cohorts from The Cancer Genome Atlas (TCGA) database. Bulk RNAseq data from primary tumor samples were evaluated for the enrichment of immunological gene signatures such as the immunologic constant of rejection, a set of 20 genes that represent the concordant activation of both innate and adaptive responses downstream of immune-mediated tissue destruction. Similar gene expression signatures have been found to be predictive of response to anti-PD-1 therapy (Ayers M, Lunceford J, Nebozhyn M, et al. J Clin Invest. 2017; 127(8):2930-40). Additional signatures representing T cells and PD-1 signaling were also evaluated (Hendrickx W, Simeone I, Anjum S, et al. Oncoimmunology. 2017; 6(2):e1253654; Bindea G, Mlecnik B, Tosolini M, et al. Immunity. 2013; 39(4):782-95; Yoshihara K, Shahmoradgoli M, Martinez E, et al. Nat Commun. 2013; 4:2612; and Quigley M, Pereyra F, Nilsson B, et al. Nat Med. 2010; 16(10):1147-51).

Samples were then evaluated for stromal and TGF-β1-related gene signatures, including the signature identified by Mariathasan et al to predict a lack of response to atezolizumab. Finally, samples were evaluated for GARP (LRRC32) expression. The resulting gene expression profiles of pancreatic adenocarcinoma, urothelial cancer (UC), hepatic cell carcinoma (HCC), head and neck squamous cell carcinoma (FINSCC), microsatellite stable colorectal cancer (MSS-CRC) and non-small cell lung cancer (NSCLC) suggest that there is an overlap between markers of T-cell infiltration, which may correlate with responsiveness to anti-PD-1 therapy, and TGF-β1-related gene signatures, indicating that release of TGF-β1 may be a mechanism of immune escape in these patients. In these tumor indications, GARP (LRRC32) expression is correlated with TGF-β1-related gene signatures, suggesting that blocking GARP-TGF-β1 may modulate the TGF-β1-related gene signatures.

In some embodiments, subjects having cancers, such as solid tumors, that evade host immunosurveillance at least partially through the expression and release of active TGF-β1 are treated with the combination of Ab1 and an anti-PD-1 antibody. In embodiments, subjects having cancers that evade host immunosurveillance at least partially through the expression and release of active TGF-β1 are administered a therapeutically effective amount of an anti-PD-1 antibody on a therapeutically effective schedule and a therapeutically effective amount on antibody that binds to a complex of hGARP and TGF-β1 on a schedule sufficient to improve the therapeutic efficacy of the anti-PD-1 antibody. In certain embodiments, the antibody that binds to a complex of hGARP and TGF-β1 is Ab1. In certain embodiments Ab1 is ABBV-151. In certain embodiments Ab1 is livmoniplimab administered at a dose ranging from about 200 mg to about 1500 mg once every two weeks, once every three weeks, or once every four weeks and the anti-PD-1 antibody is budigalimab administered at a dose of 375 mg once every three weeks, or 500 mg once every four weeks.

In embodiments, the administration to subjects having cancers that evade host immunosurveillance at least partially through the expression and release of active TGF-β1 achieves an ORR greater than that obtained with administration of the anti-PD-1 antibody alone. In embodiments, the administration to subjects having cancers that evade host immunosurveillance at least partially through the expression and release of active TGF-β1 achieves an ORR greater than or equal to 5%, greater than or equal to 10%, greater than or equal to 15%, greater than or equal to 20%, greater or equal to 25%, greater than or equal to 30%, greater than or equal to 40%, greater than or equal to 50%, greater than or equal to 60%, or greater than or equal to 70%.

In some embodiments, Ab1 treatment is added to an anti-PD-1 therapy for that cancer, such that any subject administered an anti-PD-1 antibody (e.g., an antibody that binds PD-1 or PD-L1) for treatment of that cancer is also administered Ab1.

In some embodiments, the subject has not received systemic treatment for their cancer, i.e. has not received first line systemic treatment. In embodiments, the subject has progressed after receiving first line systemic treatment. In embodiments, the subject has a relapsed or refractory cancer. In some embodiments, the subject has acquired resistance to therapy with a checkpoint inhibitor. In embodiments, the subject has acquired resistance to therapy with one or more of a PD-1 inhibitor or a PD-L1 inhibitor. In embodiments, the subject has not been treated with a checkpoint inhibitor, i.e., is check point inhibitor naïve. In embodiments, the subject has not previously received therapy with one or more of a PD-1 inhibitor or a PD-L1 inhibitor.

In some embodiments, tumors that historically do not respond to immunotherapy agents, i.e., cold tumors, such as pancreatic cancer and microsatellite stable colorectal cancer are treated with the combination of Ab1 and an anti-PD-1 antibody to achieve an ORR greater than or equal to 5%, greater than or equal to 10%, greater than or equal to 15%, greater than or equal to 20%, greater or equal to 25%, greater than or equal to 30%, greater than or equal to 40%, greater than or equal to 50%, greater than or equal to 60%, or greater than or equal to 70%. In an embodiment, the Ab1 is ABBV-151 and the anti-PD-1 antibody is ABBV-181. In an embodiment, the Ab1 is livmoniplimab and the anti-PD-1 antibody is budigalimab.

In other embodiments, inflamed or hot tumors, such as urothelial cancer (UC), HCC, HNSCC, and NSCLC are treated with the combination of Ab1 and an anti-PD-1 antibody to achieve an ORR of greater than or equal to 5%, greater than or equal to 10%, greater than or equal to 15%, greater than or equal to 20%, greater or equal to 25%, greater than or equal to 30%, greater than or equal to 40%, greater than or equal to 50%, greater than or equal to 60%, or greater than or equal to 70%. In an embodiment, the Ab1 is ABBV-151 and the anti-PD-1 antibody is ABBV-181. In an embodiment, the Ab1 is livmoniplimab and the anti-PD-1 antibody is budigalimab.

In embodiments, the cancer is a solid tumor selected from the group consisting of pancreatic adenocarcinoma, urothelial cancer (UC), including muscle invasive urothelial cancer (MIUC), hepatocellular carcinoma (HCC), head and neck squamous cell carcinoma, colorectal cancer (CRC, including microsatellite stable (MSS-CRC), non-small cell lung cancer (NSCLC), ovarian cancer, ovarian granulosa cell tumor cancer (GCT), breast cancer, or gastroesophageal junction adenocarcinoma that is treated with the combination of Ab1 and the anti-PD-1 to achieve an ORR of greater than or equal to 5%, greater than or equal to 10%, greater than or equal to 15%, greater than or equal to 20%, greater or equal to 25%, greater than or equal to 30%, greater than or equal to 40%, greater than or equal to 50%, greater than or equal to 60%, or greater than or equal to 70%. In an embodiment, the Ab1 is ABBV-151 and the anti-PD-1 antibody is ABBV-181. In an embodiment, the Ab1 is livmoniplimab and the anti-PD-1 antibody is budigalimab.

In embodiments, the combination of Ab1 and the anti-PD-1 antibody is used to treat a cancer selected from the group consisting of pancreatic adenocarcinoma, urothelial cancer, including muscle invasive urothelial cancer, hepatocellular carcinoma (HCC), head and neck squamous cell carcinoma, colorectal cancer (CRC, including microsatellite stable (MSS-CRC), non-small cell lung cancer (NSCLC), ovarian cancer, ovarian granulosa cell tumor cancer, breast cancer, or gastroesophageal junction adenocarcinoma that has metastasized to achieve an ORR of greater than or equal to 5%, greater than or equal to 10%, greater than or equal to 15%, greater than or equal to 20%, greater or equal to 25%, greater than or equal to 30%, greater than or equal to 40%, greater than or equal to 50%, greater than or equal to 60%, or greater than or equal to 70%. In an embodiment, the Ab1 is ABBV-151 and the anti-PD-1 antibody is ABBV-181. In an embodiment, the Ab1 is livmoniplimab and the anti-PD-1 antibody is budigalimab.

Provided herein is a method of treating cancer in subjects in need thereof, comprising administering a therapeutically effective amount of the combination of 1) an anti-PD-1 antibody and 2) Ab1, wherein the cancer is selected from the group consisting of muscle invasive urothelial cancer, hepatocellular carcinoma, microsatellite stable colorectal cancer, non-small cell lung cancer, and ovarian granulosa cell tumor cancer.

In an embodiment, the Ab1 in this section (Section 6.3) is ABBV-151 and the anti-PD-1 antibody is ABBV-181. In an embodiment, the Ab1 in this section (Section 6.3) is livmoniplimab and the anti-PD-1 antibody is budigalimab.

Table 3 provide examples of subjects having cancer that are treated with the combination of Ab1 and an anti-PD-1 antibody, therapeutically effective dosing regimens of the antibodies, and examples of overall response rates (ORR) for those regimens. In embodiments, sub-populations of individuals with the indicated cancer are selected and treated, with the selection being based on one or more of the listed selection criteria below each cancer. Criteria inconsistent with one another (e.g., treatment naïve vs. progression after prior therapy) are not combined for the purposes of defining sub-populations.

TABLE 3

| Subject cancer | | |
|---|---|---|
| Optional selection criteria | Dosing Regimen(s) | ORR |
| Cancers that evade host | 1. Ab1 400 mg Q3W | Greater than the |
| immunosurveillance at least | Anti-PD-1 antibody 375 mg Q3W | standard of care, |
| partially through the | 2. Ab1 600 mg Q3W | for example: |
| expression and release of | Anti-PD-1 antibody 375 mg Q3W | ≥5% |
| active TGF-β1 | 3. Ab1 800 mg Q3W | ≥8% |
| Standard of Care (SoC) | Anti-PD-1 antibody 375 mg Q3W | ≥10% |
| naïve | 4. Ab1 1200 mg Q3W | ≥15% |
| Progression during or | Anti-PD-1 antibody 375 mg Q3W | ≥20% |
| after 1 or more prior | 5. Ab1 500 mg Q4W | ≥35% |
| therapies | Anti-PD-1 antibody 500 mg Q4W | ≥40% |
| | 6. Ab1 600 mg Q4W | ≥45% |
| | Anti-PD-1 antibody 500 mg Q4W | |
| | 7. Ab1 1000 mg Q4W | |
| | Anti-PD-1 antibody 500 mg Q4W | |
| | 8. Ab1 1500 mg Q4W | |
| | Anti-PD-1 antibody 500 mg Q4W | |
| | 9. Ab1 1500 mg Q2W | |
| | Anti-PD-1 antibody 500 mg Q4W | |
| | 10. Ab1 1200 mg Q2W | |
| | Anti-PD-1 antibody 500 mg Q4W | |
| Pancreatic adenocarcinoma | 1. Ab1 400 mg Q3W | Greater than the |
| Standard of Care (SoC) | Anti-PD-1 antibody 375 mg Q3W | standard of care, |
| naive, in combination | 2. Ab1 600 mg Q3W | for example: |
| with nab-paclitaxel and | Anti-PD-1 antibody 375 mg Q3W | ≥5% |
| gemcitabine. | 3. Ab1 800 mg Q3W | ≥8% |
| Progression during or | Anti-PD-1 antibody 375 mg Q3W | ≥10% |
| after 1 systemic therapy | 4. Ab1 1200 mg Q3W | ≥15% |
| (gemcitabine | Anti-PD-1 antibody 375 mg Q3W | ≥20% |
| monotherapy or in | 5. Ab1 500 mg Q4W | ≥35% |
| combination with other | Anti-PD-1 antibody 500 mg Q4W | ≥40% |
| agents, FOLFIRINOX | 6. Ab1 600 mg Q4W | ≥45% |
| [or another regimen | Anti-PD-1 antibody 500 mg Q4W | |
| including both 5- | 7. Ab1 1000 mg Q4W | |
| fluorouracil and | Anti-PD-1 antibody 500 mg Q4W | |
| oxaliplatin], | 8. Ab1 1500 mg Q4W | |
| capecitabine | Anti-PD-1 antibody 500 mg Q4W | |
| monotherapy or in | 9. Ab1 1500 mg Q2W | |
| combination with other | Anti-PD-1 antibody 500 mg Q4W | |
| agents) administered in | 10. Ab1 1200 mg Q2W | |
| the adjuvant, locally | Anti-PD-1 antibody 500 mg Q4W | |
| advanced, or metastatic | | |
| setting. | | |
| No progression on more | | |
| than 1 prior systemic | | |
| therapy | | |
| If the therapy was used | | |
| in an adjuvant setting, | | |
| disease progression must | | |
| have occurred within 6 | | |
| months of completing | | |
| adjuvant therapy | | |
| No prior exposure to | | |
| PD-1/PD-L1 antagonist | | |
| Urothelial cancer | 1. Ab1 400 mg Q3W | Greater than the |
| Standard of Care (SoC) | Anti-PD-1 antibody 375 mg Q3W | standard of care, |
| naive | 2. Ab1 600 mg Q3W | for example: |
| Progressed following | Anti-PD-1 antibody 375 mg Q3W | ≥5% |
| treatment with a | 3. Ab1 800 mg Q3W | ≥8% |
| platinum-based regimen | Anti-PD-1 antibody 375 mg Q3W | ≥10% |
| (administered in any line | 4. Ab1 1200 mg Q3W | ≥15% |
| of therapy) and a PD- | Anti-PD-1 antibody 375 mg Q3W | ≥20% |
| 1/PD-L1 antagonist | 5. Ab1 500 mg Q4W | ≥35% |
| administered in the | Anti-PD-1 antibody 500 mg Q4W | ≥40% |
| recurrent or metastatic | 6. Ab1 600 mg Q4W | ≥45% |
| setting | Anti-PD-1 antibody 500 mg Q4W | |
| | 7. Ab1 1000 mg Q4W | |
| | Anti-PD-1 antibody 500 mg Q4W | |
| | 8. Ab1 1500 mg Q4W | |
| | Anti-PD-1 antibody 500 mg Q4W | |
| | 9. Ab1 1500 mg Q2W | |
| | Anti-PD-1 antibody 500 mg Q4W | |
| | 10. Ab1 1200 mg Q2W | |
| | Anti-PD-1 antibody 500 mg Q4W | |
| Muscle invasive urothelial | 1. Ab1 400 mg Q3W | Greater than the |
| cancer | Anti-PD-1 antibody 375 mg Q3W | standard of care, |
| Standard of Care (SoC) | 2. Ab1 600 mg Q3W | for example: |
| naive | Anti-PD-1 antibody 375 mg Q3W | ≥5% |
| ECOG 0-1 | 3. Ab1 800 mg Q3W | ≥8% |
| MIBC pT2-PT4a or | Anti-PD-1 antibody 375 mg Q3W | ≥10% |
| ypT4 | 4. Ab1 1200 mg Q3W | ≥15% |
| Radical cystectomy | Anti-PD-1 antibody 375 mg Q3W | ≥20% |
| within past 120 days | 5. Ab1 500 mg Q4W | ≥35% |
| Positive ctDNA post | Anti-PD-1 antibody 500 mg Q4W | ≥40% |
| radical cysectomy | 6. Ab1 600 mg Q4W | ≥45% |
| | Anti-PD-1 antibody 500 mg Q4W | |
| | 7. Ab1 1000 mg Q4W | |
| | Anti-PD-1 antibody 500 mg Q4W | |
| | 8. Ab1 1500 mg Q4W | |
| | Anti-PD-1 antibody 500 mg Q4W | |
| | 9. Ab1 1500 mg Q2W | |
| | Anti-PD-1 antibody 500 mg Q4W | |
| | 10. Ab1 1200 mg Q2W | |
| | Anti-PD-1 antibody 500 mg Q4W | |
| Hepatocellular carcinoma front | 1. Ab1 400 mg Q3W | Greater than the |
| line | Anti-PD-1 antibody 375 mg Q3W | standard of care, |
| Standard of Care (SoC) | 2. Ab1 600 mg Q3W | for example: |
| naive | Anti-PD-1 antibody 375 mg Q3W | ≥5% |
| Tyrosine kinase inhibitor | 3. Ab1 800 mg Q3W | ≥8% |
| (TKI)-naïve | Anti-PD-1 antibody 375 mg Q3W | ≥10% |
| No untreated brain | 4. Ab1 1200 mg Q3W | ≥15% |
| metastases | Anti-PD-1 antibody 375 mg Q3W | ≥20% |
| No prior exposure to | 5. Ab1 500 mg Q4W | ≥35% |
| PD-1/PD-L1 antagonist | Anti-PD-1 antibody 500 mg Q4W | ≥40% |
| | 6. Ab1 600 mg Q4W | ≥45% |
| | Anti-PD-1 antibody 500 mg Q4W | |
| | 7. Ab1 1000 mg Q4W | |
| | Anti-PD-1 antibody 500 mg Q4W | |
| | 8. Ab1 1500 mg Q4W | |
| | Anti-PD-1 antibody 500 mg Q4W | |
| | 9. Ab1 1500 mg Q2W | |
| | Anti-PD-1 antibody 500 mg Q4W | |
| | 10. Ab1 1200 mg Q2W | |
| | Anti-PD-1 antibody 500 mg Q4W | |
| Hepatocellular carcinoma second | 1. Ab1 400 mg Q3W | Greater than the |
| line | Anti-PD-1 antibody 375 mg Q3W | standard of care, |
| Standard of Care (SoC) | 2. Ab1 600 mg Q3W | for example: |
| naive | Anti-PD-1 antibody 375 mg Q3W | ≥5% |
| Received 1^{st} line SoC | 3. Ab1 800 mg Q3W | ≥8% |
| including an approved | Anti-PD-1 antibody 375 mg Q3W | ≥10% |
| Checkpoint Inhibitor | 4. Ab1 1200 mg Q3W | ≥15% |
| (CPI) | Anti-PD-1 antibody 375 mg Q3W | ≥20% |
| No progression on more | 5. Ab1 500 mg Q4W | ≥35% |
| than 1 prior systemic | Anti-PD-1 antibody 500 mg Q4W | ≥40% |
| therapy | 6. Ab1 600 mg Q4W | ≥45% |
| TKI-naïve | Anti-PD-1 antibody 500 mg Q4W | |
| No untreated brain | 7. Ab1 1000 mg Q4W | |
| metastases | Anti-PD-1 antibody 500 mg Q4W | |
| No prior exposure to | 8. Ab1 1500 mg Q4W | |
| PD-1/PD-L1 antagonist | Anti-PD-1 antibody 500 mg Q4W | |
| | 9. Ab1 1500 mg Q2W | |
| | Anti-PD-1 antibody 500 mg Q4W | |
| | 10. Ab1 1200 mg Q2W | |
| | Anti-PD-1 antibody 500 mg Q4W | |
| Head and neck squamous cell | 1. Ab1 400 mg Q3W | Greater than the |
| carcinoma | Anti-PD-1 antibody 375 mg Q3W | standard of care, |
| Standard of Care (SoC) | 2. Ab1 600 mg Q3W | for example: |
| naïve | Anti-PD-1 antibody 375 mg Q3W | ≥5% |
| Front line in | 3. Ab1 800 mg Q3W | ≥8% |
| combination with the | Anti-PD-1 antibody 375 mg Q3W | ≥10% |
| SoC | 4. Ab1 1200 mg Q3W | ≥15% |
| Cancer arising from the | Anti-PD-1 antibody 375 mg Q3W | ≥20% |
| oral cavity, oropharynx, | 5. Ab1 500 mg Q4W | ≥35% |
| hypopharynx, or larynx | Anti-PD-1 antibody 500 mg Q4W | ≥40% |
| Progressed following | 6. Ab1 600 mg Q4W | ≥45% |
| treatment with platinum- | Anti-PD-1 antibody 500 mg Q4W | |
| based regimen and a PD- | 7. Ab1 1000 mg Q4W | |
| 1/PD-L1 antagonist | Anti-PD-1 antibody 500 mg Q4W | |
| administered in the | 8. Ab1 1500 mg Q4W | |
| recurrent or metastatic | Anti-PD-1 antibody 500 mg Q4W | |
| setting | 9. Ab1 1500 mg Q2W | |
| | Anti-PD-1 antibody 500 mg Q4W | |
| | 10. Ab1 1200 mg Q2W | |
| | Anti-PD-1 antibody 500 mg Q4W | |
| Microsatellite stable colorectal | 1. Ab1 400 mg Q3W | Greater than the |
| cancer | Anti-PD-1 antibody 375 mg Q3W | standard of care, |
| (unselected) | 2. Ab1 600 mg Q3W | for example: |
| Standard of Care (SoC) | Anti-PD-1 antibody 375 mg Q3W | ≥5% |
| naive | 3. Ab1 800 mg Q3W | ≥8% |
| Received prior | Anti-PD-1 antibody 375 mg Q3W | ≥10% |
| fluorouracil-based | 4. Ab1 1200 mg Q3W | ≥15% |
| combination | Anti-PD-1 antibody 375 mg Q3W | ≥20% |
| chemotherapy regimens | 5. Ab1 500 mg Q4W | ≥35% |
| including oxaliplatin and | Anti-PD-1 antibody 500 mg Q4W | ≥40% |
| irinotecan | 6. Ab1 600 mg Q4W | ≥45% |
| No prior exposure to | Anti-PD-1 antibody 500 mg Q4W | |
| PD-1/PD-L1 antagonist | 7. Ab1 1000 mg Q4W | |
| | Anti-PD-1 antibody 500 mg Q4W | |
| | 8. Ab1 1500 mg Q4W | |
| | Anti-PD-1 antibody 500 mg Q4W | |
| | 9. Ab1 1500 mg Q2W | |
| | Anti-PD-1 antibody 500 mg Q4W | |
| | 10. Ab1 1200 mg Q2W | |
| | Anti-PD-1 antibody 500 mg Q4W | |
| Microsatellite stable colorectal | 1. Ab1 400 mg Q3W | Greater than the |
| cancer | Anti-PD-1 antibody 375 mg Q3W | standard of care, |
| (CMS4 enriched) | 2. Ab1 600 mg Q3W | for example: |
| Standard of Care (SoC) | Anti-PD-1 antibody 375 mg Q3W | ≥5% |
| naive | 3. Ab1 800 mg Q3W | ≥8% |
| CMS4 subtype | Anti-PD-1 antibody 375 mg Q3W | ≥10% |
| determined by NGS of | 4. Ab1 1200 mg Q3W | ≥15% |
| tumor biopsy | Anti-PD-1 antibody 375 mg Q3W | ≥20% |
| Received prior | 5. Ab1 500 mg Q4W | ≥35% |
| fluorouracil-based | Anti-PD-1 antibody 500 mg Q4W | ≥40% |
| combination | 6. Ab1 600 mg Q4W | ≥45% |
| chemotherapy regimens | Anti-PD-1 antibody 500 mg Q4W | |
| including oxaliplatin and | 7. Ab1 1000 mg Q4W | |
| irinotecan | Anti-PD-1 antibody 500 mg Q4W | |
| | 8. Ab1 1500 mg Q4W | |
| | Anti-PD-1 antibody 500 mg Q4W | |
| | 9. Ab1 1500 mg Q2W | |
| | Anti-PD-1 antibody 500 mg Q4W | |
| | 10. Ab1 1200 mg Q2W | |
| | Anti-PD-1 antibody 500 mg Q4W | |
| Relapsed/Refractory non-small | 1. Ab1 400 mg Q3W | Greater than the |
| cell lung cancer | Anti-PD-1 antibody 375 mg Q3W | standard of care, |
| Standard of Care (SoC) | 2. Ab1 600 mg Q3W | for example: |
| naive | Anti-PD-1 antibody 375 mg Q3W | ≥5% |
| One prior line of | 3. Ab1 800 mg Q3W | ≥8% |
| chemotherapy and one | Anti-PD-1 antibody 375 mg Q3W | ≥10% |
| prior anti PD-1 or anti | 4. Ab1 1200 mg Q3W | ≥15% |
| PD-L1 antibody | Anti-PD-1 antibody 375 mg Q3W | ≥20% |
| treatment | 5. Ab1 500 mg Q4W | ≥35% |
| Prior chemotherapy and | Anti-PD-1 antibody 500 mg Q4W | ≥40% |
| immunotherapy in the | 6. Ab1 600 mg Q4W | ≥45% |
| neo-adjuvant/adjuvant | Anti-PD-1 antibody 500 mg Q4W | |
| setting included, but not | 7. Ab1 1000 mg Q4W | |
| subjects who have | Anti-PD-1 antibody 500 mg Q4W | |
| progressed on more than | 8. Ab1 1500 mg Q4W | |
| one line of | Anti-PD-1 antibody 500 mg Q4W | |
| chemotherapy in the | 9. Ab1 1500 mg Q2W | |
| metastatic setting and/or | Anti-PD-1 antibody 500 mg Q4W | |
| more than one prior anti | 10. Ab1 1200 mg Q2W | |
| PD-1 or anti PD-L1 in | Anti-PD-1 antibody 500 mg Q4W | |
| the metastatic setting | | |
| No subjects with known | | |
| EGFR mutations or | | |
| ALK/ROS1 gene | | |
| rearrangements | | |
| First line non-small cell lung | 1. Ab1 400 mg Q3W | Greater than the |
| cancer with liver metastasis | Anti-PD-1 antibody 375 mg Q3W | standard of care, |
| enrichment and high PD-L1 | 2. Ab1 600 mg Q3W | for example: |
| expression (TPS ≥50%) | Anti-PD-1 antibody 375 mg Q3W | ≥5% |
| Standard of Care (SoC) | 3. Ab1 800 mg Q3W | ≥8% |
| naive | Anti-PD-1 antibody 375 mg Q3W | ≥10% |
| Subjects with | 4. Ab1 1200 mg Q3W | ≥15% |
| histologically or | Anti-PD-1 antibody 375 mg Q3W | ≥20% |
| cytologically confirmed | 5. Ab1 500 mg Q4W | ≥35% |
| advanced or metastatic | Anti-PD-1 antibody 500 mg Q4W | ≥40% |
| NSCLC | 6. Ab1 600 mg Q4W | ≥45% |
| NSCLC with high | Anti-PD-1 antibody 500 mg Q4W | |
| expression PD-L1 as | 7. Ab1 1000 mg Q4W | |
| defined by Tumor | Anti-PD-1 antibody 500 mg Q4W | |
| Proportion Score (TPS) | 8. Ab1 1500 mg Q4W | |
| ≥50%, | Anti-PD-1 antibody 500 mg Q4W | |
| no known EGFR | 9. Ab1 1500 mg Q2W | |
| mutations or ALK gene | Anti-PD-1 antibody 500 mg Q4W | |
| rearrangements, | 10. Ab1 1200 mg Q2W | |
| no prior therapy in the | Anti-PD-1 antibody 500 mg Q4W | |
| advanced or metastatic | | |
| setting | | |
| First Line Non Small Cell lung | 1. Ab1 400 mg Q3W | Greater than the |
| cancer | Anti-PD-1 antibody 375 mg Q3W | standard of care, |
| Standard of Care (SoC) | 2. Ab1 600 mg Q3W | for example: |
| naive | Anti-PD-1 antibody 375 mg Q3W | ≥5% |
| Patient population: all | 3. Ab1 800 mg Q3W | ≥8% |
| comers (regardless of | Anti-PD-1 antibody 375 mg Q3W | ≥10% |
| PD-L1 status) | 4. Ab1 1200 mg Q3W | ≥15% |
| In addition to receiving | Anti-PD-1 antibody 375 mg Q3W | ≥20% |
| Ab1 and an anti-PD-1 | 5. Ab1 500 mg Q4W | ≥35% |
| antibody, the | Anti-PD-1 antibody 500 mg Q4W | ≥40% |
| combination will also | 6. Ab1 600 mg Q4W | ≥45% |
| include the platinum | Anti-PD-1 antibody 500 mg Q4W | |
| doublet regimen for | 7. Ab1 1000 mg Q4W | |
| NSCLC: | Anti-PD-1 antibody 500 mg Q4W | |
| 75 mg per | 8. Ab1 1500 mg Q4W | |
| square meter of | Anti-PD-1 antibody 500 mg Q4W | |
| body-surface | 9. Ab1 1500 mg Q2W | |
| area or | Anti-PD-1 antibody 500 mg Q4W | |
| carboplatin (area | 10. Ab1 1200 mg Q2W | |
| under the | Anti-PD-1 antibody 500 mg Q4W | |
| concentration- | | |
| time curve, 5 mg | | |
| per milliliter per | | |
| minute) plus | | |
| pemetrexed (500 | | |
| mg per square | | |
| meter), all | | |
| administered | | |
| intravenously | | |
| every 3 weeks, | | |
| followed by | | |
| optional | | |
| pemetrexed | | |
| maintenance | | |
| (500 mg per | | |
| square meter) | | |
| every 3 weeks. | | |
| Subject has | | |
| histologically- or | | |
| cytologically-confirmed | | |
| metastatic non- | | |
| squamous NSCLC | | |
| Subject has documented | | |
| PD L1 status | | |
| Subject has not received | | |
| prior systemic therapy | | |
| Ovarian Granulosa Cell tumor | 1. Ab1 400 mg Q3W | Greater than the |
| containing | Anti-PD-1 antibody 375 mg Q3W | standard of care, |
| FOXL2 C134W mutation | 2. Ab1 600 mg Q3W | for example: |
| Standard of Care (SoC) | Anti-PD-1 antibody 375 mg Q3W | ≥5% |
| naive | 3. Ab1 800 mg Q3W | ≥8% |
| | Anti-PD-1 antibody 375 mg Q3W | ≥10% |
| | 4. Ab1 1200 mg Q3W | ≥15% |
| | Anti-PD-1 antibody 375 mg Q3W | ≥20% |
| | 5. Ab1 500 mg Q4W | ≥35% |
| | Anti-PD-1 antibody 500 mg Q4W | ≥40% |
| | 6. Ab1 600 mg Q4W | ≥45% |
| | Anti-PD-1 antibody 500 mg Q4W | |
| | 7. Ab1 1000 mg Q4W | |
| | Anti-PD-1 antibody 500 mg Q4W | |
| | 8. Ab1 1500 mg Q4W | |
| | Anti-PD-1 antibody 500 mg Q4W | |
| | 9. Ab1 1500 mg Q2W | |
| | Anti-PD-1 antibody 500 mg Q4W | |
| | 10. Ab1 1200 mg Q2W | |
| | Anti-PD-1 antibody 500 mg Q4W | |

In an embodiment, the Ab1 in Table 3 is ABBV-151 and the anti-PD-1 antibody is ABBV-181. In an embodiment, the Ab1 in Table 3 is livmoniplimab and the anti-PD-1 antibody is budigalimab. The selection of doses is informed by the proposed mechanism of action of livmoniplimab, clinical efficacy and safety, clinical PK/PD modeling, and preclinical evidence demonstrating livmoniplimab's concentration dependent ability to inhibit active TGF-β1 release from the GARP-TGF-β1 complex expected to inhibit subsequent signaling within the TME.

### 7. EXAMPLES

### 7.1. Example 1: Study Objectives for the M19-345 Phase 1 First-In Human, Multi-Center, Open Label Dose-Escalation Study to Determine the Safety, Tolerability, Pharmacokinetics and RP2D of ABBV-151 as a Single Agent and in Combination with ABBV-181 in Subjects with Locally Advanced or Metastatic Solid Tumors

This is a Phase 1, open-label, dose-escalation, dose-expansion, PK, biomarker/PD, and proof-of-activity study. The study will assess the safety, PK, PD, and preliminary efficacy of ABBV-151 as monotherapy and in combination with budigalimab.

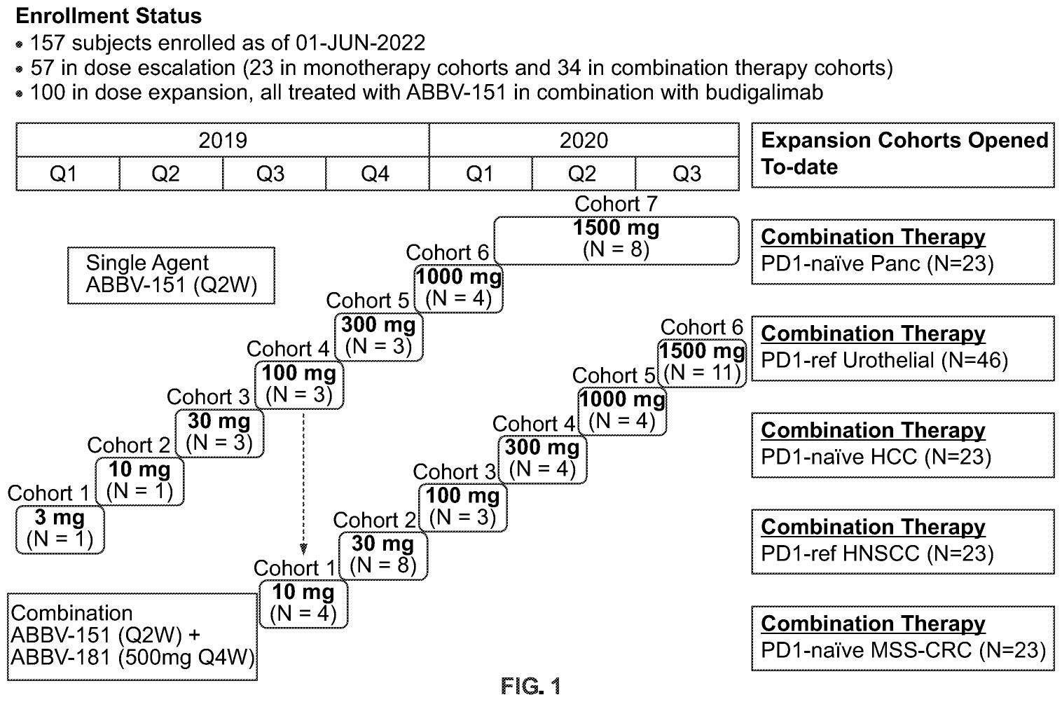

Approximately 257 subjects with locally advanced or metastatic solid tumors will be enrolled in this FIH study. This trial will consist of 2 parts as shown in FIG. 1 and described below. Approximately 46 subjects with solid tumors will be enrolled in the dose escalation cohorts. Approximately 138-191 subjects with pancreatic adenocarcinoma, urothelial cancer, HCC, HNSCC, MSS-CRC, and NSCLC will be enrolled into the expansion cohorts.

Subjects will receive ABBV-151 and/or budigalimab (combination cohorts only) until disease progression or intolerable toxicity.

7.1.1. Dose Escalation Cohorts:

Dose escalation will be the FIH evaluation of ABBV-151 as a single agent administered in ascending dose cohorts guided by a Bayesian optimal interval (BOIN) design. The ABBV-151 monotherapy dose escalation arm will be initiated first. ABBV-151 will be administered by 60-minute intravenous (IV) infusion Q2W. Eligible subjects will have an advanced solid tumor who are considered refractory to or intolerant of all existing therapy(ies) known to provide a clinical benefit for their condition (i.e., subjects who have progressed on standard therapies known to provide clinical benefit). A cycle is defined as 28 days. The monotherapy dose escalation will lead to the characterization of the safety profile, PK profile and target engagement, and to the selection of the monotherapy RP2D for ABBV-151 to be used as described below. Efficacy data will be collected as an exploratory endpoint during dose escalation.

The combination therapy dose escalation arm of ABBV-151 and budigalimab will begin once the first 2 or more dose levels of ABBV-151 monotherapy have been declared safe. The starting dose of ABBV-151 in combination with budigalimab will be at least 2 dose levels below the highest ABBV-151 monotherapy dose level shown to be safe and at least 3 subjects will be treated with ABBV-151 monotherapy prior to the start of the combination dose escalation. The dose escalation combination of ABBV-151 and budigalimab will be guided by the BOIN design with a minimum cohort size of 3 subjects. The dose of budigalimab will be fixed at 500 mg (flat dosing) via IV infusion Q4W.

7.1.2. Eligibility Criteria:

Adult subjects with an advanced solid tumor who are considered refractory to or intolerant of all existing therapy(ies) known to provide a clinical benefit for their condition (i.e., subjects who have progressed on standard therapies known to provide clinical benefit). Additionally, subjects who have been offered standard therapies and refused, or who are considered ineligible for standard therapies, may be eligible for this study on a case-by-case basis. Subjects with pancreatic adenocarcinoma, urothelial cancer, HCC, or HNSCC who are being considered for the dose escalation cohorts must also meet the histology specific eligibility criteria described below for dose expansion.

7.1.3. Dose Expansion Cohorts:

Dose expansion will further assess the safety and tolerability of ABBV-151 given at the RP2D determined in Dose Escalation administered in combination with budigalimab. All dose expansion arms will only begin after the RP2D/MTD or MAD has been defined for both ABBV-151 monotherapy and ABBV-151+budigalimab combination therapy.

The RP2D selected for dose expansion is 1500 mg ABBV-151 Q2W administered as monotherapy or in combination with budigalimab.

Dose expansion will include 6 cohorts with 6 tumor types under evaluation (pancreatic adenocarcinoma, urothelial cancer, HCC, HNSCC, MSS-CRC, and NSCLC). The expansion cohorts will evaluate the following:
ABBV-151 in combination with budigalimab in pancreatic adenocarcinoma
ABBV-151 in combination with budigalimab in urothelial cancer
ABBV-151 in combination with budigalimab in HCC
ABBV-151 in combination with budigalimab in HNSCC
ABBV-151 in combination with budigalimab in MSS-CRC
ABBV-151 in combination with budigalimab in NSCLC

Dose expansion will provide characterization of, safety profile, PK/PD, and preliminary efficacy for ABBV-151 in combination with budigalimab.

7.1.4. Eligibility Criteria:

All subjects with HCC, pancreatic adenocarcinoma, or MSS-CRC must not have had prior exposure to a prior PD-1/PD-L1 antagonist in any line of therapy.

Pancreatic adenocarcinoma subjects must have disease progression during or after 1 systemic therapy (gemcitabine monotherapy or in combination with other agents, FOLFIRINOX [or another regimen including both 5-fluorouracil and oxaliplatin], capecitabine monotherapy or in combination with other agents) administered in the adjuvant, locally advanced, or metastatic setting. Progression on more than 1 prior systemic therapy is not allowed in this cohort. If the therapy was used in an adjuvant setting, disease progression must have occurred within 6 months of completing adjuvant therapy.

Urothelial cancer of the bladder and urinary tract subjects must have progressed following treatment with a platinum-based regimen (administered in any line of therapy) and a PD-1/PD-L1 antagonist administered in the recurrent or metastatic setting (progression following a PD-1/PD-L1 antagonist is defined as unequivocal progression on or within 3 months of the last dose of anti-PD-1 or anti-PD-L1 therapy).

Hepatocellular carcinoma subjects must have disease progression during or after 1 prior line of systemic therapy. Progression on more than 1 prior systemic therapy is not allowed in this cohort. Subjects must have a Child-Pugh A classification and must not have ascites that requires chronic therapy (i.e., not requiring diuretics, repeat paracenteses, or an indwelling catheter). Subjects with varices are eligible as long as they received appropriate prophylaxis/intervention per local guidelines. Subjects must also meet specific requirements regarding viral hepatitis status.

Head and neck squamous cell carcinoma (arising from the oral cavity, oropharynx, hypopharynx, or larynx) subjects must have progressed following treatment with platinum-based regimen (administered in any line of therapy) and a PD-1/PD-L1 antagonist administered in the recurrent or metastatic setting (progression following a PD-1/PD-L1 antagonist is defined as unequivocal progression on or within 3 months of the last dose of anti-PD-1 or anti-PD-L1 therapy).

CRC subjects with microsatellite stable or mismatch repair proficient colorectal adenocarcinoma (as determined by PCR/NGS or IHC, respectively) who have received prior fluorouracil-based combination chemotherapy regimens including oxaliplatin and irinotecan (with or without VEGF and/or EGFR targeted agents).

Subjects with histologically or cytologically confirmed advanced or metastatic NSCLC who have received 1 prior line of chemotherapy and 1 prior anti-PD-(L)1 antibody, administered either concurrently or sequentially in the metastatic setting. Prior chemotherapy and immunotherapy in the neo-adjuvant/adjuvant setting is allowed, but subjects who have progressed on more than 1 line of chemotherapy in the metastatic setting and/or more than 1 prior anti-PD-(L)1 in the metastatic setting will not be eligible. Progression following a PD-1/PD-L1 antagonist is defined as unequivocal progression on or within 3 months of the last dose of anti-PD-1 or anti-PD-L1 therapy. NSCLC subjects with known EGFR mutations or ALK/ROS1 gene rearrangements are ineligible.

Subjects must also have:

An Eastern Cooperative Oncology Group (ECOG) Performance Status of 0 to 1, adequate bone marrow, renal, hepatic, and coagulation function. Must not have received anticancer therapy including chemotherapy, immunotherapy, radiation therapy, biologic, herbal therapy, or any investigational therapy within a period of 5 half-lives or 28 days (whichever is shorter), prior to the first dose of the study drug. Have no unresolved AEs >Grade 1 from prior anticancer therapy except for alopecia. No clinically significant uncontrolled condition(s); no active bacterial, fungal, or viral infections; and no active autoimmune disease, with exceptions of vitiligo, type I diabetes mellitus, hypothyroidism, and psoriasis. No history of primary immunodeficiency, bone marrow transplantation, solid organ transplantation, or previous clinical diagnosis of tuberculosis. No history of inflammatory bowel disease, interstitial lung disease or pneumonitis, myocarditis, Stevens-Johnson syndrome, toxic epidermal necrolysis or drug reaction with eosinophilia and systemic symptoms (DRESS). No known uncontrolled metastases to the central nervous system (with certain exceptions).

Viral Hepatitis Status for all subjects WITHOUT HCC: must confirm that subject tests negative for active hepatitis A, B, or C.

No current or prior use of immunosuppressive medication within 14 days prior to the first dose of the study drug.

No live vaccine administration ≤28 days prior to the first dose of study drug.

### 7.2. Example 2: Results from the M19-345 Phase 1 First-In Human, Multi-Center, Open Label Dose-Escalation Study to Determine the Safety, Tolerability, Pharmacokinetics and RP2D of ABBV-151 as a Single Agent and in Combination with ABBV-181 in Subjects with Locally Advanced or Metastatic Solid Tumors

7.2.1. Summary:

As of Jun. 1, 2022, 157 subjects have been enrolled. Of those subjects, 57 were in the dose escalation cohorts, 23 in the monotherapy cohort and 34 in the combination therapy cohort. In the dose expansion cohort, 100 subjects have been enrolled and treated with the combination of ABBV-151+budigalimab.

The dose escalation enrolled subjects with advanced solid tumors considered refractory to or intolerant of all existing therapies known to provide a clinical benefit for their condition. In the monotherapy dose escalation cohort, 23 subjects were enrolled who received seven dose levels of ABBV-151 as monotherapy ranging from 3 mg to 1500 mg administered intravenously every 2 weeks (Q2W). The combination dose escalation enrolled 34 subjects who received six dose levels of ABBV-151, ranging from 10 mg to 1500 mg Q2W in combination with a fixed dose of the anti-PD-1 antibody, budigalimab (500 mg Q4W). The RP2D selected of ABBV-151 was determined to be 1500 mg every two weeks (Q2W) as monotherapy or in combination with budigalimab.

The objective response rate was 0% for subjects treated with monotherapy and was 12% in the combination dose escalation. The response rate regardless of confirmation was 0% in the monotherapy dose escalation and 15% in the combination dose escalation, and an additional 26.5% of subjects treated with combination therapy had a best response of stable disease. Subjects enrolled in the dose escalation included both those who had received anti-PD-1 therapy and those who were PD-1 naïve, and included several tumor types including non-small cell lung cancer, ovarian cancer, pancreatic adenocarcinoma, breast cancer (both triple negative breast cancer and hormone receptor positive breast cancer), colorectal cancer, urothelial carcinoma, endometrial cancer, renal cell carcinoma, gastric and gastroesophageal junction cancer, prostate adenocarcinoma, uterine adenocarcinoma, mesothelioma, hemangiopericytoma, and several less common adenocarcinomas, carcinomas, and sarcomas.

The dose expansion cohorts enrolled subjects treated with ABBV-151+budigalimab combination therapy. The cancer types included PD-1 relapsed/refractory urothelial cancer, PD-1 relapsed/refractory head and neck squamous cell carcinomas (HNSCC) and PD-1 relapsed/refractory non-small cell lung cancer (NSCLC), and PD-1 naive microsatellite stable colorectal cancer (MSS-CRC), PD-1 naive hepatocellular carcinoma (HCC), and PD-1 naïve pancreatic adenocarcinoma and ovarian granulosa cell tumor.

Subjects that responded to treatment with ABBV-151+budigalimab included 1 subject with gastroesophageal junction adenocarcinoma, 4 subjects with colorectal cancer (3 out of four with MSS-CRC), 2 subjects with ovarian cancer (granulosa subtype), 1 subject with pancreatic adenocarcinoma, 7 subjects with urothelial carcinoma, and 5 subjects with hepatocellular carcinoma and 3 subjects with ovarian granulosa cell tumor. Several additional subjects experienced durable stable disease for 6 months or greater. Accordingly, the combination of ABBV-151+budigalimab demonstrates durable anti-tumor activity in heavily pretreated PD-1 relapsed and refractory subjects and also in PD-1 naïve subjects.

7.2.2. ABBV-151 Monotherapy Dose Escalation Results:

Twenty-three subjects were enrolled in the monotherapy escalation cohort. Sixty five percent were anti-PD-(L)1 naïve and had received 4 median prior lines of therapy. The tumor types included 4 NSCLC, 3 Ovarian, 1 Pancreatic, 3 CRC, 2 TNBC, 1 Breast (non TNBC) and 9 other solid tumors (Endometrial (N=2), Osteosarcoma, Mesothelioma, Stomach (N=2), Rhabdomyosarcoma, Papillary adenosarcoma hemangiopericytoma).

The results are shown in FIG. 2. There were no responders in the monotherapy cohort. The objective response rate (ORR) was 0%, the best overall response rate (includes unconfirmed) was 0%, and durable stable disease (SD) for approximately 6 months or more was 0.

7.2.3. ABBV-151+ABBV-181 Dose Escalation Combination Therapy Results:

Thirty-four subjects were enrolled in the combination therapy dose escalation cohort. Seventy percent were anti PD-(L)1 naïve and had received 3 median prior lines of therapy. Tumor types included: 1 NSCLC, 7 Ovarian, 4 Pancreatic, 8 CRC, 1 Urothelial, 2 Breast (non TNBC), 11 other solid tumors (Renal cell carcinoma, Adrenocortical carcinoma, Prostate adenocarcinoma, Gastroesophageal junction adenocarcinoma, Sebaceous carcinoma (Orbital sebaceous gland cancer), Uterine adenocarcinoma, Leiomyosarcoma, Ampullary adenocarcinoma, Clear cell sarcoma, Alveolar soft part sarcoma, and Endometrial adenocarcinoma).

The results are shown in FIG. 2. The objective response rate was 4/34 (11.8%) and the best overall response rate (includes unconfirmed) was 5/34 (14.7%). Additional subjects who did not respond but had durable stable disease for approximately 6 months or more was 4/34 (11.8%).

The responders included 1 gastroesophageal junction adenocarcinoma subject who was PD-1 naïve (20004, 30 mg ABBV-151 combo cohort), two colorectal cancer subjects (12010, who was PD-1 naïve and treated in the 30 mg ABBV-151 combo cohort, and 20007, who had prior PD-1 inhibitor treatment and treated in the 100 mg ABBV-151 combo cohort), and a PD-1-naïve ovarian cancer subject (10015, treated in the 1500 mg ABBV-151 combo cohort). One subject with PD-1-naïve ovarian cancer achieved an unconfirmed PR at the last disease assessment (10017, treated in the 1500 mg ABBV-151 combo cohort). An additional 4 subjects have had stable disease for 6 months or longer as of the data cutoff (12007 with PD-1-relapsed colorectal cancer in the 10 mg ABBV-151 combo cohort, 40007 with PD-1-naïve alveolar sarcoma in the 1500 mg ABBV-151 combo cohort, 10019 with PD-1-naïve ovarian cancer in the 1500 mg ABBV-151 combo cohort, and 30012 with PD-1 relapsed urothelial cancer in the 1500 mg ABBV-151 combo cohort.

7.2.4. ABBV-151+ABBV-181 Dose Expansion—PD-1 R/R Urothelial Carcinoma:

Subjects were enrolled with histologically or cytologically confirmed urothelial cancer of the bladder and urinary tract who had progressed following treatment with a platinum-based regimen (administered in any line of therapy) and a PD-1/PD-L1 antagonist administered in the recurrent or metastatic setting (progression following a PD-1/PD-L1 antagonist is defined as unequivocal progression on or within 3 months of the last dose of anti-PD-1 or anti-PDL-1 therapy).

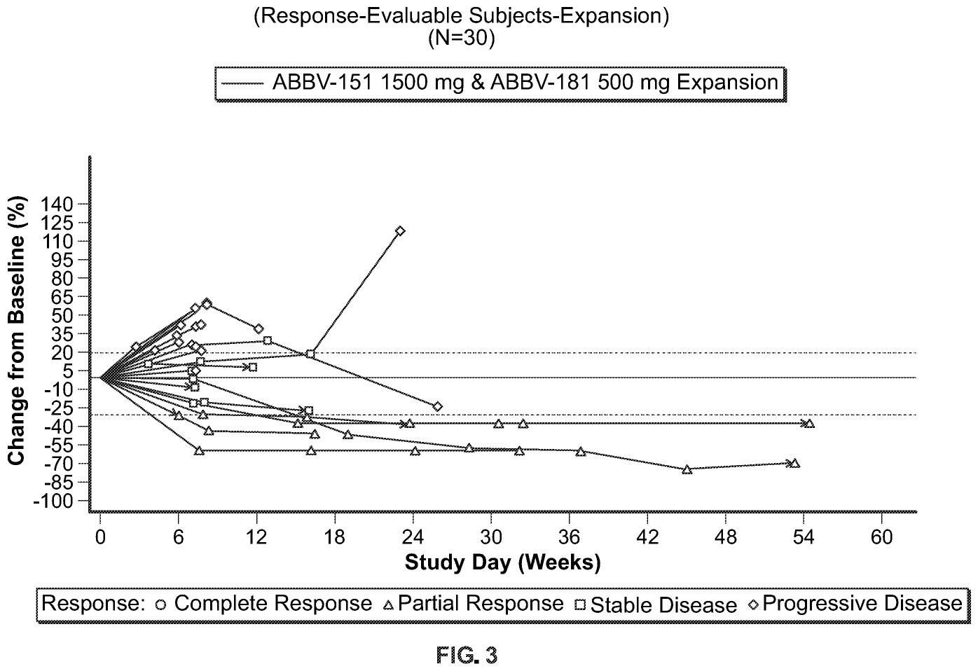

As of May 2022, 32 have been enrolled, with 3 median prior lines of therapy, including a few that had prior enfortumab vedotin, including 1 responder who failed prior EV. The results are shown in FIG. 3. The objective response rate to date is 5/32 (15.6%). The best overall response rate (includes unconfirmed) to date is 6/32 (18.8%). As of July 26, there are 7 responders (6 confirmed) out of 36 enrolled. In addition, 1 additional subject had durable stable disease for approximately 6 months or more.

As of Mar. 30, 2023, 48 patients have been enrolled, 45 were response evaluable, with a confirmed ORR by RECIST 1.1 of 18%.

7.2.5. ABBV-151+ABBV-181 Dose Expansion—PD-1 Naïve Hepatocellular Carcinoma (HCC):

Subjects were enrolled with histologically confirmed advanced HCC who had disease progression during or after 1 prior line of systemic therapy. Progression on more than 1 prior systemic therapy is not allowed in this cohort. Subjects must have a Child-Pugh A classification and must not have ascites that requires chronic therapy. Subjects with varices are eligible as long as they have been received appropriate prophylaxis/intervention per local guidelines. Additional viral status eligibility in the protocol.

Twelve subjects have been enrolled, mostly with prior sorafenib treatment but a few with prior lenvatinib or other tyrosine kinase inhibitors (TKIs). Median prior lines of therapy is 1.

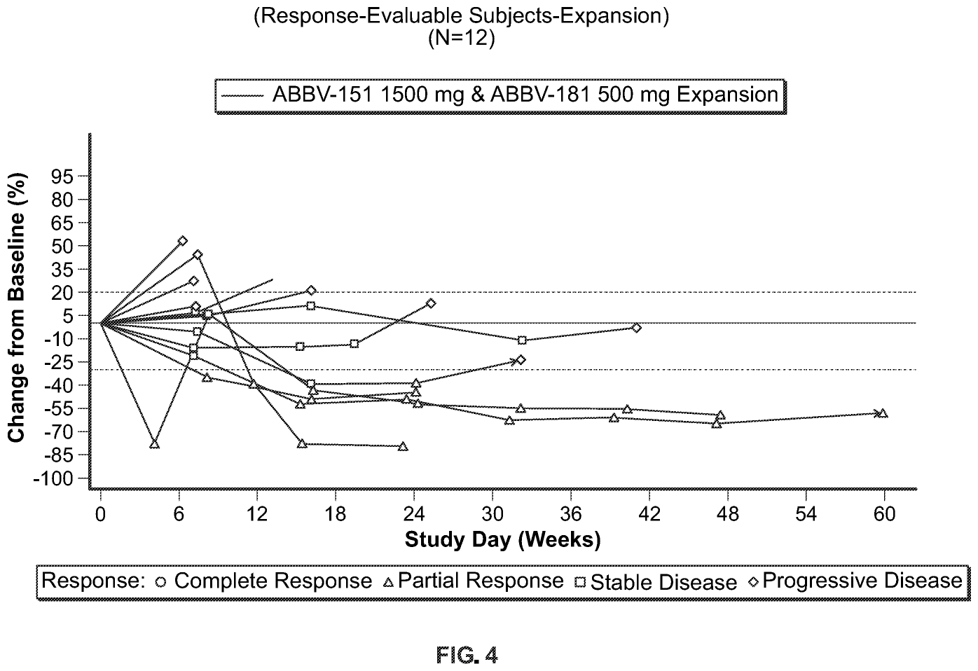

The results are shown in FIG. 4. The objective response rate is 3/12 (25%). As of July 2022 there are now 5 confirmed responders. The best overall response rate (includes unconfirmed) is 4/12 (33.3%). Two additional subjects had durable stable disease for approximately 6 months or more.

As of Mar. 30, 2023, 12 patients have been enrolled, all were response evaluable, with a confirmed ORR by iRECIST of 42%.

7.2.6. ABBV-151+ABBV-181 Dose Expansion— PD-1 Naïve Pancreatic Adenocarcinoma:

Subjects were enrolled with histologically or cytologically confirmed advanced or metastatic pancreatic adenocarcinoma who had disease progression during or after 1 systemic therapy. As of May 2022, 23 subjects have been enrolled with 2 median prior lines of therapy.

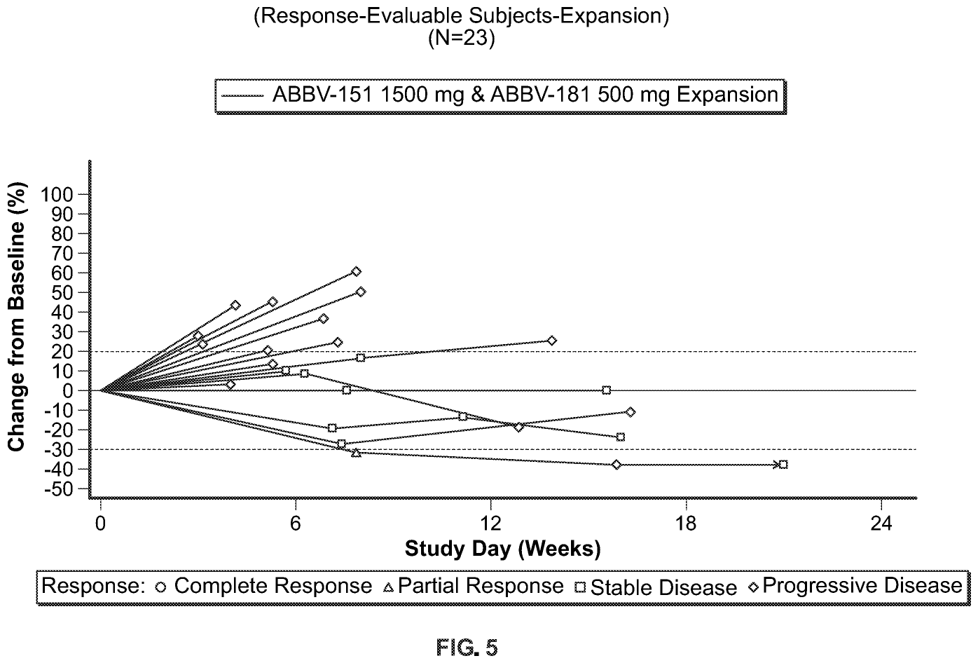

The results are shown in FIG. 5. The objective response rate is 0%. The best overall response rate (includes unconfirmed) is 1/23 (4.3%). One additional subject had durable stable disease for approximately 6 months.

As of Mar. 30, 2023, 23 patients have been enrolled, all were response evaluable, with a confirmed ORR by RECIST 1.1 of 0%.

7.2.7. ABBV-151+ABBV-181 Dose Expansion— PD-1 Naïve Microsatellite Stable Colorectal Adenocarcinoma:

Subjects were enrolled with microsatellite stable or mismatch repair proficient colorectal adenocarcinoma (as determined by PCR/NGS or IHC, respectively) who had received 1-2 prior chemotherapy regimens and who had refused or are ineligible for other approved therapies. Subjects with progression on more than 2 prior systemic therapies will not be eligible for this cohort. Subjects must have historical microsatellite instability or mismatch repair test results available or have available archival tissue suitable for prospective testing at Pre-Screening. Subjects known to have a high tumor mutational burden (defined as ≥10 mutations/megabase) based on historical results will not be eligible.

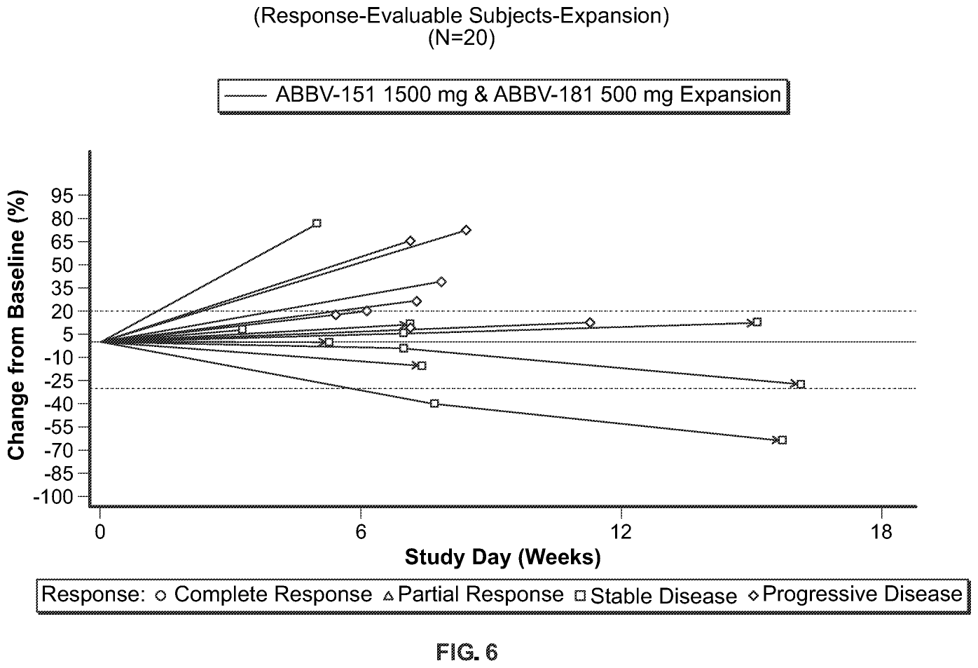

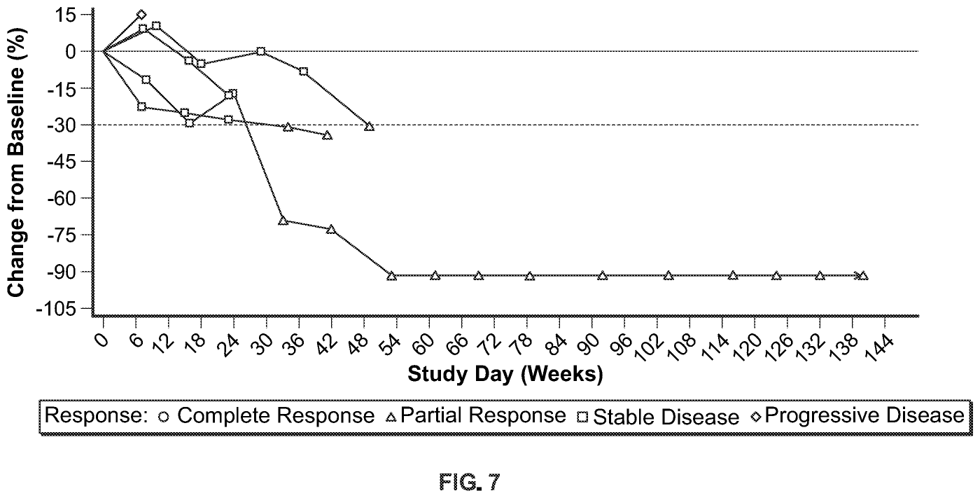

The results are shown in FIG. 6. As of July 2022, 25 subjects have been enrolled. The objective response rate is 1/25 (4%). The best overall response rate (includes unconfirmed) is 2/25 (8%). One additional subject had durable stable disease for approximately 6 months.

As of Mar. 30, 2023, 25 patients have been enrolled, 24 were response evaluable, with a confirmed ORR by RECIST 1.1 of 8%.

7.2.8. ABBV-151+ABBV-181 Dose Expansion—NSCLC

As of Mar. 30, 2023, 3 patients have been enrolled, all 3 were response evaluable, with a confirmed ORR of 0%. This cohort only recently started enrolling, thus the results are immature and more patient data is required to fully evaluate the efficacy of the combination in NSCLC.

7.2.9. ABBV-151+ABBV-181 Dose Expansion—Ovarian Granulosa Cell Tumor

As of Mar. 30, 2023, 4 patients have been enrolled, the unconfirmed ORR is 75%.

### 7.3. Example 3: PK/PD Analysis, Modeling, and Dose Optimization

7.3.1. Pharmacodynamic Biomarker

GARP/TGF-β1 target engagement of ABBV-151 on activated platelets from clinical samples was determined using a validated method.

7.3.2. Pharmacokinetics and Pharmacodynamics

Pharmacokinetic samples were obtained at specified visits and timepoints. Serum concentrations were determined using a validated method for ABBV-151.

A nonlinear mixed-effects modeling approach was used to estimate the population PK parameters of ABBV-151 such as clearance (CL), and volume (V). An Eₘₐₓ model was used to model the pharmacodynamics to estimate concentration needed to achieve 95% of platelet GARP/TGF-β1 target engagement (EC₉₅) in circulation and subsequently extrapolation to the tumor microenvironment.

7.3.3. Clinical PK/PD Modeling and Dose Optimization

Livmoniplimab is administered as monotherapy and combination with budigalimab was well tolerated with no major safety concerns across doses tested in the dose escalation (Study M19-345). The maximum administered dose (MAD) is 1500 mg Q2W in combination with budigalimab and is being evaluated in the expansion phase across multiple solid tumor indications. Clinical responses (confirmed responses per RECIST criteria) were observed at as low as 30 mg, Q2W in combination with budigalimab during dose escalation and at 1500 mg, Q2W in dose expansion.

Based on preclinical and clinical PK/PD assessments, doses predicted to provide sufficient pharmacological activity at the tumor site and potentially clinical efficacy in majority of the subjects in the population treated include 500 mg, Q4W and above or 375 mg, Q3W and above. As evidenced by clinical activity during dose escalation portion and expansion portion of M19-345, lower doses may also be effective.

The identification of a dose range that is pharmacologically and clinically active was guided by achieving specific target concentrations at the tumor site: (1) C_{min,C1} values (28-day cycle for Q4W and 21-day cycle for Q3W) needed to achieve greater than or equal to the upper limit of 95% prediction interval (PI) of EC₉₅ for GARP/TGF-β1 target engagement in the tumor microenvironment for majority of the subjects based on clinical PK/PD data. (2) Minimum C_{min,C1} needed to maximally inhibit release of TGF-β1 from the GARP-TGF-β1 complex and subsequently inhibition of autocrine and paracrine signaling of TGF-β1 at the tumor microenvironment. This target concentration was informed by preclinical in vitro assay which demonstrated livmoniplimab at the minimal concentration of 0.8 ug/mL maximally inhibited TGF-β1 release and signaling.

Table 4 summarizes the percentage of subjects at the indicated Q4W dosages achieving these two target concentrations at the tumor site. The results from Q3W dosing that also results in similar exposure for Q4W regimen are summarized in Table 5. As shown livmoniplimab 500 mg, Q4W or 375 mg, Q3W would enable ≥95% of the subjects to achieve complete target saturation and blockage of TGF-β1 release and signaling at the tumor site including for subjects at the lower end of PI of livmoniplimab predicted tumor exposure (C_{min,C1}). Thus, livmoniplimab 500 mg Q4W or 375 mg Q3W is the predicted minimal dose required for maximal pharmacological activity across solid tumor indications in majority of subjects treated starting from Cycle 1 and doses below 500 mg, Q4W or 375 mg Q3W may result in insufficient exposure in fraction of subjects treated in Cycle 1 that could compromise the goal of providing pharmacological activity and potentially the clinical efficacy as soon as possible.

In addition to the model predicted active dose ranges (500 mg, Q4W and above; 375 mg Q3W and above), the observed clinical active dose range of 30 mg Q2W and above will be investigated.

TABLE 4
Predicted Serum and Tumor Livmoniplimab Trough Concentrations Following Q4W Dosing (28-day Cycle) and
Percentage of Subjects with Median Cₘᵢₙ, C1 Achieving Upper Limit of 95% PI of EC₉₅ for GARP/TGF-β1 Target
Engagement and Minimally Required Concentration to Completely Block TGF-β1 Release and Signaling

| | | | | | | | % of Subjects with Median Tumor | |
|---|---|---|---|---|---|---|---|---|
| | Cycle 1 Trough Concentration | | | | % of Subjects with Median Tumor | | C_{min, C1} ≥ Minimally Required | |
| | Predicted Median | | Predicted Median | | C_{min, C1} ≥ Upper Limit of 95% of | | Concentration to Completely Block | |
| | Serum C_{min, C1} | | Tumor C_{min, C1} | | CI EC₉₅ for Target Engagement | | TGF-β1 Release and Signaling | |
| Dose (mg) | [95% PI] (μg/mL) | | [95% PI] (μg/mL)^{a} | | [95% PI] (0.5 μg/mL)^{b} | | [95% PI] (0.8 μg/mL)^{c} | |
| 250 | 14.7 | [6.29, 26.7] | 0.737 | [0.314, 1.33] | 80.3 | [79.99, 80.68] | 41.9 | [41.51, 42.37] |
| 250^{d} | 14.21 | [5.625, 26.63] | 0.7104 | [0.2812, 1.332] | 76.55 | [76.17, 76.92] | 38.82 | [38.40, 39.25] |
| 500 | 33.7 | [15.5, 60.4] | 1.68 | [0.777, 3.02] | 99.0 | [98.86, 99.04] | 94.5 | [94.27, 94.67] |
| 500^{d} | 30.86 | [12.72, 57.71] | 1.543 | [0.6358, 2.885] | 97.59 | [97.45, 97.72] | 90.12 | [89.86, 90.38] |
| 750^{d} | 47.89 | [20.18, 90.47] | 2.395 | [1.009, 4.524] | 99.41 | [99.34, 99.47] | 97.504 | [97.36, 97.64] |
| 1000^{d} | 66.27 | [28.28, 122.7] | 3.313 | [1.414, 6.136] | 99.83 | [99.79, 99.85] | 99.224 | [99.14, 99.30] |
| 1500 | 106 | [50.5, 188] | 5.32 | [2.53, 9.42] | 100 | [99.99, 100] | 99.9 | [99.91, 99.95] |
| 1500^{d} | 98.43 | [42.01, 184] | 4.921 | [2.101, 9.20] | 99.94 | [99.92, 99.96] | 99.766 | [99.72, 99.81] |

^{a}Predicted tumor livmoniplimab concentrations were based on assumption of at least 5% tumor penetration of monoclonal antibodies reported in clinical studies. Guolan Lu et al. Predicting Therapeutic Antibody Delivery into Human Head and Neck Cancers. Clin Cancer Res 2020; 26: 2582-94; Andrew M. Scott et al. APhase I Trial of Humanized Monoclonal Antibody A33 in Patients with Colorectal Carcinoma: Biodistribution, Pharmacokinetics, and QuantitativeTumor Uptake. Clin Cancer Res 2005; 11: 4810-17; Li et al. Clin Pharmacol Ther. 2021 July; 110(1): 200-209
^{b}Estimated EC₉₅ for target engagement was based on preliminary clinical PK/Pharmacodynamic modeling of GARP/TGF-β1 target engagement data on platelets from clinical samples.
^{c}In vitro potency of livmoniplimab to block TGF-β1 release from the GARP-TGF-β1 complex measured by a reporter assay in human GARP-TGF-β1 expressed on HEK293T cells.
^{d}Predictions utilizing an updated model, dose selection consistent between original and updated models.

TABLE 5
Predicted Serum and Tumor Livmoniplimab Trough Concentrations Following Q3W Dosing (21-day Cycle) and
Percentage of Subjects with Median Cₘᵢₙ, C1 Achieving Upper Limit of 95% PI of EC₉₅ for GARP/TGF-β1 Target
Engagement and Minimally Required Concentration to Completely Block TGF-β1 Release and Signaling

| | | | | | | | % of Subjects with Median Tumor | |
|---|---|---|---|---|---|---|---|---|
| | Cycle 1 Trough Concentration | | | | % of Subjects with Median Tumor | | C_{min, C1} ≥ Minimally Required | |
| | Predicted Median | | Predicted Median | | C_{min, C1} ≥ Upper Limit of 95% of | | Concentration to Completely Block | |
| | Serum C_{min, C1} | | Tumor C_{min, C1} | | CI EC₉₅ for Target Engagement | | TGF-β1 Release and Signaling | |
| Dose (mg) | [95% PI] (μg/mL) | | [95% PI] (μg/mL)^{a} | | [95% PI] (0.5 μg/mL)^{b} | | [95% PI] (0.8 μg/mL)^{c} | |
| 200 | 15.1 | [7.71, 26.1] | 0.754 | [0.386, 1.30] | 85.8 | [85.48, 86.10] | 43.4 | [42.97, 43.83] |
| 200^{d} | 14.5 | [7.04, 25.7] | 0.726 | [0.352, 1.28] | 82.2 | [81.8, 82.5] | 39.6 | [39.2, 40.0] |
| 375 | 30.5 | [16.1, 51.5] | 1.53 | [0.802, 2.58] | 99.5 | [99.40, 99.53] | 95.1 | [94.89, 95.26] |
| 400^{d} | 31.3 | [15.5, 54.6] | 1.57 | [0.774, 2.73] | 99.2 | [99.1, 99.2] | 94.3 | [94.1, 94.5] |
| 800^{d} | 66.3 | [33.1, 117] | 3.31 | [1.65, 5.83] | 100 | [99.9, 100] | 99.8 | [99.7, 99.8] |
| 1125 | 96.2 | [52.1, 161] | 4.81 | [2.61, 8.07] | 100 | [99.99, 100] | 100 | [99.97, 99.99] |
| 1200^{d} | 99.5 | [49.5, 175] | 4.97 | [2.48, 8.75] | 100 | [100, 100.0] | 100 | [99.9, 100] |

^{a}Predicted tumor livmoniplimab concentrations were based on assumption of at least 5% tumor penetration of monoclonal antibodies reported in clinical studies. Guolan Lu et al. Predicting Therapeutic Antibody Delivery into Human Head and Neck Cancers. Clin Cancer Res 2020; 26: 2582-94; Andrew M. Scott et al. APhase I Trial of Humanized Monoclonal Antibody A33 in Patients with Colorectal Carcinoma: Biodistribution, Pharmacokinetics, and QuantitativeTumor Uptake. Clin Cancer Res 2005; 11: 4810-17; Li et al. Clin Pharmacol Ther. 2021 July; 110(1): 200-209
^{b}Estimated EC₉₅ for target engagement was based on preliminary clinical PK/Pharmacodynamic modeling of GARP/TGF-β1 target engagement data on platelets.
^{c}In vitro potency of livmoniplimab to block TGF-β1 release from the GARP-TGF-β1 complex measured by a reporter assay in human GARP-TGF-β1 expressed on HEK293T cells.
^{d}Predictions utilizing an updated model, dose selection consistent between original and updated models.

### 8. EXEMPLARY EMBODIMENTS

While various specific embodiments have been illustrated and described, and some are represented below, it will be appreciated that various changes can be made without departing from the spirit and scope of the inventions(s).

### 8.1. TGF-β1 Expressing Solid Tumor Embodiments

1. A method of treating a cancer that evades host immunosurveillance at least partially through the expression and release of active TGF-β1, comprising administering to a human subject having said tumor the combination of
a) a therapeutically effective amount an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose ranging from about 200 mg to about 1600 mg once every two weeks, once every three weeks.
2. The method of embodiment 1, wherein the anti-TGF-β1/GARP complex antibody is administered once every two weeks at a dose ranging from about 200 mg to about 1500 mg.
3. The method of embodiment 1, wherein the anti-TGF-β1/GARP complex antibody is administered once every three weeks at a dose ranging from about 200 mg to about 1200 mg.
4. The method of embodiment 1, wherein the anti-TGF-β1/GARP complex antibody is administered once every four weeks at a dose ranging from about 200 mg to about 1600 mg.
5. The method of embodiment 1, wherein the anti-TGF-β1/GARP complex antibody is administered at a dose selected from the group consisting of 200 mg, 400 mg, 500 mg, 600 mg, 800 mg, 1000 mg, 1200 mg, 1500 mg, and 1600 mg.
6. The method of embodiment 2, wherein the anti-TGF-β1/GARP complex antibody is administered at a dose of 1500 mg.
7. The method of embodiment 3, wherein the anti-TGF-β1/GARP complex antibody is administered at a dose of 400 mg.
8. The method of embodiment 3, wherein the anti-TGF-β1/GARP complex antibody is administered at a dose of 600 mg.
9. The method of embodiment 3, wherein the anti-TGF-β1/GARP complex antibody is administered at a dose of 1200 mg.
10. The method of embodiment 4, wherein the anti-TGF-β1/GARP complex antibody is administered at a dose of 500 mg.
11. The method of embodiment 4, wherein the anti-TGF-β1/GARP complex antibody is administered at a dose of 600 mg.
12. The method of embodiment 4, wherein the anti-TGF-β1/GARP complex antibody is administered at a dose of 1500 mg.
13. A method of treating a cancer that evades host immunosurveillance at least partially through the expression and release of active TGF-β1, comprising administering to a human subject having said cancer the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose selected from the group consisting of 200 mg, 400 mg, 500 mg, 600 mg, 800 mg and 1200 mg once every three weeks.
14. The method of embodiment 12, wherein the anti-TGF-β1/GARP complex antibody is administered at a dose of 400 mg.
15. The method of embodiment 12, wherein the anti-TGF-β1/GARP complex antibody is administered at a dose of 600 mg.
16. The method of embodiment 12, wherein the anti-TGF-β1/GARP complex antibody is administered at a dose of 1200 mg.
17. A method of treating a cancer that evades host immunosurveillance at least partially through the expression and release of active TGF-β1, comprising administering to a human subject having said cancer the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose selected from the group consisting of 200 mg, 400 mg, 500 mg, 550 mg, 600 mg, 1000 mg, 1500 mg, and 1600 mg once every four weeks.
18. The method of embodiment 17, wherein the anti-TGF-β1/GARP complex antibody is administered at a dose of 500 mg.
19. The method of embodiment 17, wherein the anti-TGF-β1/GARP complex antibody is administered at a dose of 600 mg.
20. The method of embodiment 17, wherein the anti-TGF-β1/GARP complex antibody is administered at a dose of 1500 mg.
21. A method of treating a cancer that evades host immunosurveillance at least partially through the expression and release of active TGF-β1 in a population of human subjects, comprising administering to the human subjects having said a cancer that evades host immunosurveillance at least partially through the expression and release of active TGF-β1, the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose ranging from about 200 mg to about 1600 mg once every two weeks, once every three weeks, or once every four weeks,
wherein the overall response rate (ORR) is greater than the standard of care, such as from 5% or greater, 15% or greater, 20% or greater, 30% or greater, or 40% or greater.
22. A TGF-β1/GARP complex antibody for use in combination with an anti-PD-1 antibody in the treatment a cancer that evades host immunosurveillance at least partially through the expression and release of active TGF-β1, wherein the anti-TGF-β1/GARP complex antibody consists of two heavy chains each consisting of the amino acid sequence of SEQ ID NO:9 and two light chains each consisting of the amino acid sequence of SEQ ID NO:10, and wherein the anti-TGF-β1/GARP complex antibody is administered at a dose ranging from about 200 mg to about 1600 mg once every two weeks, once every three weeks or once every four weeks, and the anti-PD-1 antibody is administered at a dose of 375 mg once every three weeks, or 500 mg once every four weeks.

### 8.2. Hepatocellular Carcinoma Embodiments

1. A method of treating a hepatocellular carcinoma (HCC), comprising administering to a human subject having said HCC the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose ranging from about 200 mg to about 1600 mg once every two weeks, once every three weeks, or once every four weeks.
2. The method of embodiment 1, wherein the anti-TGF-β1/GARP complex antibody is administered once every two weeks at a dose ranging from about 200 mg to about 1600 mg.
3. The method of embodiment 1, wherein the anti-TGF-β1/GARP complex antibody is administered once every three weeks at a dose ranging from about 200 mg to about 1200 mg.
4. The method of embodiment 1, wherein the anti-TGF-β1/GARP complex antibody is administered once every four weeks at a dose ranging from about 200 mg to about 1600 mg.
5. The method of embodiment 1, wherein the anti-TGF-β1/GARP complex antibody is administered at a dose selected from the group consisting of 200 mg, 400 mg, 500 mg, 600 mg, 800 mg, 1000 mg, 1200 mg, 1500 mg, and 1600 mg.
6. The method of embodiment 2, wherein the anti-TGF-β1/GARP complex antibody is administered at a dose of 1500 mg.
7. The method of embodiment 3, wherein the anti-TGF-β1/GARP complex antibody is administered at a dose of 400 mg.
8. The method of embodiment 3, wherein the anti-TGF-β1/GARP complex antibody is administered at a dose of 600 mg.
9. The method of embodiment 3, wherein the anti-TGF-β1/GARP complex antibody is administered at a dose of 1200 mg.
10. The method of embodiment 4, wherein the anti-TGF-β1/GARP complex antibody is administered at a dose of 500 mg.
11. The method of embodiment 4, wherein the anti-TGF-β1/GARP complex antibody is administered at a dose of 600 mg.
12. The method of embodiment 4, wherein the anti-TGF-β1/GARP complex antibody is administered at a dose of 1500 mg.
13. The method of embodiment 1, wherein the subject having said HCC previously received a first-line treatment for HCC and experienced disease progression on the first-line treatment.
14. The method of embodiment 13, wherein the first-line treatment includes a checkpoint inhibitor.
15. The method of embodiment 1, wherein the subject having said HCC progressed on more than one prior line of treatment.
16. The method of embodiment 1, wherein the subject having said HCC received no prior treatment with a tyrosine kinase inhibitor, had no untreated brain metastases, and no prior exposure to a PD-1 or PD-L1 antagonist.
17. The method of embodiment 1, wherein the anti-TGF-β1/GARP complex antibody is administered intravenously prior to the intravenous administration of the anti-PD-1 antibody, and the anti-TGF-β1/GARP complex antibody and the anti-PD-1 antibody are administered on the same day as one another.
18. The method of embodiment 1, wherein the anti-PD-1 antibody is budigalimab.
19. The method of embodiment 2, wherein the anti-PD-1 antibody is budigalimab, administered at a dose of 500 mg once every four weeks.
20. The method of embodiment 3, wherein the anti-PD-1 antibody is budigalimab administered at a dose of 375 mg once every three weeks.
21. The method of embodiment 4, wherein the anti-PD-1 antibody is budigalimab administered at a dose of 500 mg once every four weeks.
22. The method of embodiment 1, wherein the subject having said HCC received no previous treatment for HCC.
23. A method of treating a hepatocellular carcinoma (HCC), comprising administering to a human subject having said HCC the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose selected from the group consisting of 200, 400, 500, 600 mg, 800 mg and 1200 mg once every three weeks,
wherein the subject having said HCC previously received a first-line treatment for HCC and experienced disease progression on the first-line treatment.
24. The method of embodiment 23, wherein the anti-TGF-β1/GARP complex antibody is administered at a dose of 400 mg.
25. The method of embodiment 23, wherein the anti-TGF-β1/GARP complex antibody is administered at a dose of 600 mg.
26. The method of embodiment 23, wherein the anti-TGF-β1/GARP complex antibody is administered at a dose of 1200 mg.
27. A method of treating a hepatocellular carcinoma (HCC), comprising administering to a human subject having said HCC a therapeutically effective amount of the combination of
a) an anti-PD-1 antibody
b) an anti-TGF-β1/GARP complex antibody consisting of two heavy chains each consisting of the amino acid sequence of SEQ ID NO:9 and two light chains each consisting of the amino acid sequence of SEQ ID NO:10, and
c) bevacizumab,
wherein the anti-TGF-β1/GARP complex antibody is administered at a dose selected from the group consisting of 400 mg, 600 mg, 800 mg and 1200 mg once every three weeks, the anti-PD-1 antibody is administered at a dose of 375 mg once every three weeks, and bevacizumab is administered at a dose of 15 mg/kg once every three weeks,
wherein the subject having said HCC received no previous treatment for HCC.
28. A method of treating a hepatocellular carcinoma (HCC), comprising administering to a human subject having said HCC of the combination
a) a therapeutically effective of amount an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose selected from the group consisting of 500 mg, 550 mg, 600 mg, 1000 mg, 1500 mg, and 1600 mg once every four weeks.
29. The method of embodiment 28, wherein the anti-TGF-β1/GARP complex antibody is administered at a dose of 500 mg.
30. The method of embodiment 28, wherein the anti-TGF-β1/GARP complex antibody is administered at a dose of 1000 mg.
31. The method of embodiment 28, wherein the anti-TGF-β1/GARP complex antibody is administered at a dose of 1500 mg.
32. A method of treating a hepatocellular carcinoma (HCC) in a population of human subjects, comprising administering to the human subjects having said HCC the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose ranging from about 200 mg to about 1600 mg once every two weeks, once every three weeks, or once every four weeks.
33. A TGF-β1/GARP complex antibody for use in combination with an anti-PD-1 antibody in the treatment of HCC, wherein the anti-TGF-β1/GARP complex antibody consists of two heavy chains each consisting of the amino acid sequence of SEQ ID NO:9 and two light chains each consisting of the amino acid sequence of SEQ ID NO:10, and wherein the anti-TGF-β1/GARP complex antibody is administered at a dose ranging from about 200 mg to about 1600 mg once every two weeks, once every three weeks or once every four weeks.

### 8.3. Urothelial Cancer Embodiments

1. A method of treating a urothelial cancer (UC), comprising administering to a human subject having said UC the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose ranging from about 200 mg to about 1600 mg once every two weeks, once every three weeks, or once every four weeks.
2. The method of embodiment 1, wherein the UC is muscle invasive urothelial cancer (MIUC).
3. The method of embodiment 2, wherein the MIUC is categorized as MIUC pT2-PT4a or ypT4.
4. The method of embodiment 1, wherein the anti-TGF-β1/GARP complex antibody is administered once every two weeks at a dose ranging from about 200 mg to about 1600 mg.
5. The method of embodiment 1, wherein the anti-TGF-β1/GARP complex antibody is administered once every three weeks at a dose ranging from about 200 mg to about 1200 mg.
6. The method of embodiment 1, wherein the anti-TGF-β1/GARP complex antibody is administered once every four weeks at a dose ranging from about 200 mg to about 1600 mg.
7. The method of embodiment 1, wherein the anti-TGF-β1/GARP complex antibody is administered at a dose selected from the group consisting of 200 mg, 400 mg, 500 mg, 600 mg, 800 mg, 1000 mg, 1200 mg, 1400 mg, and 1500 mg.
8. The method of embodiment 4, wherein the anti-TGF-β1/GARP complex antibody is administered at a dose of 1500 mg.
9. The method of embodiment 5, wherein the anti-TGF-β1/GARP complex antibody is administered at a dose of 400 mg.
10. The method of embodiment 5, wherein the anti-TGF-β1/GARP complex antibody is administered at a dose of 600 mg.
11. The method of embodiment 5, wherein the anti-TGF-β1/GARP complex antibody is administered at a dose of 1200 mg.
12. The method of embodiment 6, wherein the anti-TGF-β1/GARP complex antibody is administered at a dose of 500 mg.
13. The method of embodiment 6, wherein the anti-TGF-β1/GARP complex antibody is administered at a dose of 600 mg.
14. The method of embodiment 6, wherein the anti-TGF-β1/GARP complex antibody is administered at a dose of 1500 mg.
15. The method of embodiment 1, wherein the subject having said urothelial cancer or MIUC received a prior line of treatment for their cancer and experienced disease progression on the prior line treatment.
16. The method of embodiment 15, wherein the prior line of treatment is treatment with a platinum-based regimen and/or a PD-1/PD-L1 antagonist administered in the recurrent or metastatic setting.
17. The method of embodiment 1, wherein the anti-TGF-β1/GARP complex antibody and the anti-PD-1 antibody are administered intravenously on the same day as one another, and the anti-TGF-β1/GARP complex antibody is administered prior to the administration of the anti-PD-1 antibody.
18. The method of embodiment 1, wherein the anti-PD-1 antibody is budigalimab.
19. The method of embodiment 2, wherein the anti-PD-1 antibody is budigalimab administered at a dose of 500 mg once every four weeks.
20. The method of embodiment 5, wherein the anti-PD-1 antibody is budigalimab administered at a dose of 375 mg once every three weeks.
21. The method of embodiment 6, wherein the anti-PD-1 antibody is budigalimab administered at a dose of 500 mg once every four weeks.
22. The method of embodiment 1, wherein the administration of the anti-TGF-β1/GARP complex antibody and the anti-PD-1 antibody are administered intravenously.
23. The method of embodiment 1, wherein the subject having said urothelial cancer or MIUC received no previous treatment for urothelial cancer or MIUC.
24. A method of treating a urothelial cancer, comprising administering to a human subject having said cancer the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose selected from the group consisting of 200 mg, 400 mg, 500 mg, 600 mg, 800 mg and 1200 mg once every three weeks.
25. The method of embodiment 24, wherein the anti-TGF-β1/GARP complex antibody is administered at a dose of 400 mg.
26. The method of embodiment 24, wherein the anti-TGF-β1/GARP complex antibody is administered at a dose of 600 mg.
27. The method of embodiment 24, wherein the anti-TGF-β1/GARP complex antibody is administered at a dose of 1200 mg.
28. A method of treating a urothelial cancer, comprising administering to a human subject having said cancer the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose selected from the group consisting of 200 mg, 400 mg, 500 mg, 550 mg, 600 mg, 1000 mg, 1500 mg, and 1600 mg once every four weeks.
29. The method of embodiment 28, wherein the anti-TGF-β1/GARP complex antibody is administered at a dose of 500 mg.
30. The method of embodiment 28, wherein the anti-TGF-β1/GARP complex antibody is administered at a dose of 600 mg.
31. The method of embodiment 28, wherein the anti-TGF-β1/GARP complex antibody is administered at a dose of 1500 mg.
32. The method of treating a urothelial cancer in a population of human subjects, comprising administering to the human subjects having said urothelial cancer the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose ranging from about 200 mg to about 1600 mg once every two weeks, once every three weeks, or once every four weeks,
wherein the overall response rate (ORR) is greater than the standard of care, such as 5% or greater, 10% or greater, 15% or greater, or 20% or greater.
33. The method of embodiment 32, wherein the UC is MIUC.
34. A TGF-β1/GARP complex antibody for use in combination with an anti-PD-1 antibody in the treatment of urothelial cancer, wherein the anti-TGF-β1/GARP complex antibody consists of two heavy chains each consisting of the amino acid sequence of SEQ ID NO:9 and two light chains each consisting of the amino acid sequence of SEQ ID NO:10, and wherein the anti-TGF-β1/GARP complex antibody is administered at a dose ranging from about 200 mg to about 1600 mg once every two weeks, once every three weeks or once every four weeks.

### 8.4. Non-Small Cell Lung Cancer Embodiments

1. A method of treating non-small cell lung cancer (NSCLC), comprising administering to a human subject having said NSCLC the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose ranging from about 200 mg to about 1600 mg once every two weeks, once every three weeks, or once every four weeks.
2. The method of embodiment 1, wherein the combination further comprises carboplatin and pemetrexed.
3. The method of embodiment 1, wherein the anti-TGF-β1/GARP complex antibody is administered once every two weeks at a dose ranging from about 200 mg to about 1500 mg.
4. The method of embodiment 1, wherein the anti-TGF-β1/GARP complex antibody is administered once every three weeks at a dose ranging from about 200 mg to about 1200 mg.
5. The method of embodiment 1, wherein the anti-TGF-β1/GARP complex antibody is administered once every four weeks at a dose ranging from about 200 mg to about 1600 mg.
6. The method of embodiment 4, wherein the anti-TGF-β1/GARP complex antibody is administered at a dose selected from the group consisting of 200 mg, 400 mg, 500 mg, 600 mg, 800 mg, 1000 mg, 1200 mg, 1500 mg, and 1600 mg.
7. The method of embodiment 3, wherein the anti-TGF-β1/GARP complex antibody is administered at a dose of 1500 mg
8. The method of embodiment 4, wherein the anti-TGF-β1/GARP complex antibody is administered at a dose of 400 mg.
9. The method of embodiment 4, wherein the anti-TGF-β1/GARP complex antibody is administered at a dose of 600 mg.
10. The method of embodiment 4, wherein the anti-TGF-β1/GARP complex antibody is administered at a dose of 1200 mg.
11. The method of embodiment 5, wherein the anti-TGF-β1/GARP complex antibody is administered at a dose of 500 mg.
12. The method of embodiment 5, wherein the anti-TGF-β1/GARP complex antibody is administered at a dose of 600 mg.
13. The method of embodiment 5, wherein the anti-TGF-β1/GARP complex antibody is administered at a dose of 1500 mg.
14. The method of embodiment 1 or 2, wherein said administration is a first-line treatment.
15. The method of embodiment 1 or 2, wherein the NSCLC has a PD-L1 TPS≥1%.
16. The method of embodiment 1 or 2, wherein the NSCLC has a PD-L1 TPS≥50%.
17. The method of embodiment 1, wherein said subject has received more than one prior line of treatment and said subject's NSCLC is relapsed/refractory.
18. The method of embodiment 1 or 2, wherein said subject's NSCLC has a PD-L1 TPS≥50% and said subject has liver metastasis.
19. The method of embodiment 1 or 2, wherein the anti-TGF-β1/GARP complex antibody and the anti-PD-1 antibody are administered intravenously on the same day as one another, and the anti-TGF-β1/GARP complex antibody is administered prior to the administration of the anti-PD-1 antibody.
20. The method of embodiment 1 or 2, wherein the anti-PD-1 antibody is budigalimab.
21. The method of embodiment 3, wherein the anti-PD-1 antibody is budigalimab administered at a dose of 500 mg once every four weeks.
22. The method of embodiment 4, wherein the anti-PD-1 antibody is budigalimab administered at a dose of 375 mg once every three weeks.
23. The method of embodiment 5, wherein the anti-PD-1 antibody is budigalimab administered at a dose of 500 mg once every four weeks.
24. A method of treating non-small cell lung cancer (“NSCLC”), comprising administering to a human subject having said NSCLC the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose selected from the group consisting of 200 mg, 400 mg, 500 mg, 600 mg, 800 mg and 1200 mg once every three weeks.
25. The method of embodiment 24, wherein the anti-TGF-β1/GARP complex antibody is administered at a dose of 400 mg.
26. The method of embodiment 24, wherein the anti-TGF-β1/GARP complex antibody is administered at a dose of 600 mg.
27. The method of embodiment 24, wherein the anti-TGF-β1/GARP complex antibody is administered at a dose of 1200 mg.
28. A method of treating non-small cell lung cancer (“NSCLC”), comprising administering to a human subject having said NSCLC the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose selected from the group consisting of 200 mg, 400 mg, 500 mg, 550 mg, 600 mg, 1000 mg, 1500 mg, and 1600 mg once every four weeks.
29. The method of embodiment 28, wherein the anti-TGF-β1/GARP complex antibody is administered at a dose of 500 mg.
30. The method of embodiment 28, wherein the anti-TGF-β1/GARP complex antibody is administered at a dose of 600 mg.
31. The method of embodiment 28, wherein the anti-TGF-β1/GARP complex antibody is administered at a dose of 1500 mg.
32. The method of treating non-small cell lung cancer (“NSCLC”) in a population of human subjects, comprising administering to the human subjects having said NSCLC the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose ranging from about 200 mg to about 1600 mg once every two weeks, once every three weeks, or once every four weeks,
wherein the overall response rate (ORR) is greater than the standard of care, such as 20% or greater, 30% or greater, or 40% or greater.
33. The method of embodiment 32, wherein the combination further comprises carboplatin and pemetrexed.
34. A TGF-β1/GARP complex antibody for use in combination with an anti-PD-1 antibody in the treatment of NSCLC, wherein the anti-TGF-β1/GARP complex antibody consists of two heavy chains each consisting of the amino acid sequence of SEQ ID NO:9 and two light chains each consisting of the amino acid sequence of SEQ ID NO:10, and wherein the anti-TGF-β1/GARP complex antibody is administered at a dose ranging from about 200 mg to about 1600 mg once every two weeks, once every three weeks or once every four weeks.

### 8.5. Microsatellite Stable Colorectal Cancer Embodiments

1. A method of treating a microsatellite stable colorectal cancer (MSS-CRC), comprising administering to a human subject having said MSS-CRC the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose ranging from about 200 mg to about 1600 mg once every two weeks, once every three weeks, or once every four weeks.
2. The method of embodiment 1, wherein the MSS-CRC is the CMS4 subtype.
3. The method of embodiment 1, wherein the MSS-CRC is unselected for the CMS4 subtype.
4. The method of any one of embodiments 1-3, wherein the anti-TGF-β1/GARP complex antibody is administered once every two weeks at a dose ranging from about 200 mg to about 1500 mg.
5. The method of any one of embodiments 1-3, wherein the anti-TGF-β1/GARP complex antibody is administered once every three weeks at a dose ranging from about 200 mg to about 1200 mg.
6. The method of any one of embodiments 1-3, wherein the anti-TGF-β1/GARP complex antibody is administered once every four weeks at a dose ranging from about 200 mg to about 1600 mg.
7. The method of any one of embodiments 1-3, wherein the anti-TGF-β1/GARP complex antibody is administered at a dose selected from the group consisting of 200 mg, 400 mg, 500 mg, 600 mg, 800 mg, 1000 mg, 1200 mg, and 1400 mg.
8. The method of embodiment 4, wherein the anti-TGF-β1/GARP complex antibody is administered at a dose of 1500 mg.
9. The method of embodiment 5, wherein the anti-TGF-β1/GARP complex antibody is administered at a dose of 400 mg.
10. The method of embodiment 5, wherein the anti-TGF-β1/GARP complex antibody is administered at a dose of 600 mg.
11. The method of embodiment 5, wherein the anti-TGF-β1/GARP complex antibody is administered at a dose of 1200 mg.
12. The method of embodiment 6, wherein the anti-TGF-β1/GARP complex antibody is administered at a dose of 500 mg.
13. The method of embodiment 6, wherein the anti-TGF-β1/GARP complex antibody is administered at a dose of 600 mg.
14. The method of embodiment 6, wherein the anti-TGF-β1/GARP complex antibody is administered at a dose of 1500 mg.
15. The method of any one of embodiments 1-3, wherein the subject having said MSS-CRC previously received a prior line of treatment for microsatellite stable colorectal cancer and experienced disease progression on the first-line treatment.
16. The method of embodiment 15, wherein the prior line of treatment is a fluorouracil-based combination chemotherapy and the subject had no prior exposure to anti-PD-1 or anti-PD-L1 antibody treatment.
17. The method of embodiment 16, wherein the prior line of treatment included oxaliplatin or irinotecan.
18. The method of any one of embodiments 1-3, wherein the anti-TGF-β1/GARP complex antibody and the anti-PD-1 antibody are administered intravenously on the same day as one another, and the anti-TGF-β1/GARP complex antibody is administered prior to the administration of the anti-PD-1 antibody.
19. The method of any one of embodiments 1-3, wherein the anti-PD-1 antibody is budigalimab.
20. The method of embodiment 4, wherein the anti-PD-1 antibody is budigalimab administered at a dose of 500 mg once every four weeks.
21. The method of embodiment 5, wherein the anti-PD-1 antibody is budigalimab is administered at a dose of 375 mg once every three weeks.
22. The method of embodiment 6, wherein the anti-PD-1 antibody is budigalimab administered at a dose of 500 mg once every four weeks.
23. The method of any one of embodiments 1-3, wherein the administration of the anti-TGF-β1/GARP complex antibody and the anti-PD-1 antibody are administered intravenously.
24. The method of any one of embodiments 1-3, wherein the subject having said MSS-CRC received no previous treatment for MSS-CRC.
25. A method of treating MSS-CRC, comprising administering to a human subject having said MSS-CRC combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose selected from the group consisting of 200 mg, 400 mg, 500 mg, 600 mg, 800 mg and 1200 mg once every three weeks.
26. The method of embodiment 25, wherein the anti-TGF-β1/GARP complex antibody is administered at a dose of 400 mg.
27. The method of embodiment 25, wherein the anti-TGF-β1/GARP complex antibody is administered at a dose of 600 mg.
28. The method of embodiment 25, wherein the anti-TGF-β1/GARP complex antibody is administered at a dose of 1200 mg.
29. The method of embodiment 25, wherein the MSS-CRC is the CMS4 subtype.
30. The method of embodiment 25, wherein the MSS-CRC is unselected for the CMS4 subtype.
31. A method of treating MSS-CRC, comprising administering to a human subject having said MSS-CRC the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose selected from the group consisting of 200 mg, 400 mg, 500 mg, 550 mg, 600 mg, 1000 mg, 1500 mg, and 1600 mg once every four weeks.
32. The method of embodiment 31, wherein the anti-TGF-β1/GARP complex antibody is administered at a dose of 500 mg.
33. The method of embodiment 31, wherein the anti-TGF-β1/GARP complex antibody is administered at a dose of 600 mg.
34. The method of embodiment 31, wherein the anti-TGF-β1/GARP complex antibody is administered at a dose of 1500 mg.
35. The method of embodiment 31, wherein the MSS-CRC is the CMS4 subtype.
36. The method of embodiment 31, wherein the MSS-CRC is unselected for the CMS4 subtype.
37. A method of treating MSS-CRC in a population of human subjects, comprising administering to the human subjects having said MSS-CRC the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose ranging from about 200 mg to about 1600 mg once every two weeks, once every three weeks, or once every four weeks,
wherein the overall response rate (ORR) is greater than the standard of care, such as from 5% or greater, 15% or greater, 20% or greater, 30% or greater, or 40% or greater.
38. The method of embodiment 37, wherein the MSS-CRC is the CMS4 subtype.
39. The method of embodiment 37, wherein the MSS-CRC is unselected for the CMS4 subtype.
34. A TGF-β1/GARP complex antibody for use in combination with an anti-PD-1 antibody in the treatment of MSS-CRC, wherein the anti-TGF-β1/GARP complex antibody consists of two heavy chains each consisting of the amino acid sequence of SEQ ID NO:9 and two light chains each consisting of the amino acid sequence of SEQ ID NO:10, and wherein the anti-TGF-β1/GARP complex antibody is administered at a dose ranging from about 200 mg to about 1600 mg once every two weeks, once every three weeks or once every four weeks.

### 8.6. Ovarian Granulosa Cell Tumor Embodiments

1. A method of treating an ovarian granulosa cell tumor (GCT), comprising administering to a human subject having said GCT the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose ranging from about 200 mg to about 1600 mg once every two weeks, once every three weeks, or once every four weeks.
2. The method of embodiment 1, wherein the GCT contains a FOXL2 gene mutation.
3. The method of embodiment 2, wherein the FOXL2 mutation is a C134W mutation.
4. The method of any one of embodiments 1-3, wherein the anti-TGF-β1/GARP complex antibody is administered once every two weeks at a dose ranging from about 200 mg to about 1500 mg.
5. The method of any one of embodiments 1-3, wherein the anti-TGF-β1/GARP complex antibody is administered once every three weeks at a dose ranging from about 200 mg to about 1200 mg.
6. The method of any one of embodiments 1-3, wherein the anti-TGF-β1/GARP complex antibody is administered once every four weeks at a dose ranging from about 200 mg to about 1600 mg.
7. The method of any one of embodiments 1-3, wherein, wherein the anti-TGF-β1/GARP complex antibody is administered at a dose selected from the group consisting of 200 mg, 400 mg, 500 mg, 600 mg, 800 mg, 1000 mg, 1200 mg, 1500 mg, and 1600 mg.
8. The method of embodiment 4, wherein the anti-TGF-β1/GARP complex antibody is administered at a dose of 1500 mg.
9. The method of embodiment 5, wherein the anti-TGF-β1/GARP complex antibody is administered at a dose of 400 mg.
10. The method of embodiment 5, wherein the anti-TGF-β1/GARP complex antibody is administered at a dose of 600 mg.
11. The method of embodiment 5, wherein the anti-TGF-β1/GARP complex antibody is administered at a dose of 1200 mg.
12. The method of embodiment 6, wherein the anti-TGF-β1/GARP complex antibody is administered at a dose of 500 mg.
13. The method of embodiment 6, wherein the anti-TGF-β1/GARP complex antibody is administered at a dose of 600 mg.
14. The method of embodiment 6, wherein the anti-TGF-β1/GARP complex antibody is administered at a dose of 1500 mg.
15. The method of any one of embodiments 1-3, wherein the subject having said GCT previously underwent a unilateral salpingo-oophorectomy or bilateral salpingo-oophorectomy within the 120 days prior to treatment with the anti-TGF-β1/GARP complex antibody.
16. The method of any one of embodiments 1-3, wherein the anti-TGF-β1/GARP complex antibody and the anti-PD-1 antibody are administered intravenously on the same day as one another, and the anti-TGF-β1/GARP complex antibody is administered prior to the administration of the anti-PD-1 antibody.
17. The method of any one of embodiments 1-3, wherein the anti-PD-1 antibody is budigalimab.
18. The method of embodiment 2, wherein the anti-PD-1 antibody is budigalimab administered at a dose of 500 mg once every four weeks.
19. The method of embodiment 5, wherein the anti-PD-1 antibody is budigalimab administered at a dose of 375 mg once every three weeks.
20. The method of embodiment 6, wherein the anti-PD-1 antibody is budigalimab, and the anti-PD-1 antibody is administered at a dose of 500 mg or 600 mg once every four weeks.
21. The method of any one of embodiments 1-3, wherein the administration of the anti-TGF-β1/GARP complex antibody and the anti-PD-1 antibody are administered intravenously.
22. The method of any one of embodiments 1-3, wherein the subject having said GCT received no previous systemic treatment for GCT.
23. A method of treating an ovarian granulosa cell tumor (GCT) containing a FOXL2 C134W mutation, comprising administering to a human subject having said GCT the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose selected from the group consisting of 200 mg, 400 mg, 500 mg, 600 mg, 800 mg and 1200 mg once every three weeks,
wherein the subject having said GCT previously underwent a unilateral salpingo-oophorectomy or bilateral salpingo-oophorectomy within the 120 days prior to treatment with the anti-TGF-β1/GARP complex antibody.
24. A method of embodiment 23, wherein the anti-TGF-β1/GARP complex antibody is administered at a dose of 400 mg.
25. A method of embodiment 23, wherein the anti-TGF-β1/GARP complex antibody is administered at a dose of 600 mg.
26. A method of embodiment 23, wherein the anti-TGF-β1/GARP complex antibody is administered at a dose of 1200 mg.
27. A method of an ovarian granulosa cell tumor (GCT) containing a FOXL2 C134W mutation, comprising administering to a human subject having said GCT the combination of
a) a therapeutically effective amount of an anti-PD-1 antibody
b) an anti-TGF-β1/GARP complex antibody consisting of two heavy chains each consisting of the amino acid sequence of SEQ ID NO:9 and two light chains each consisting of the amino acid sequence of SEQ ID NO:10, and
c) bevacizumab,
wherein the anti-TGF-β1/GARP complex antibody is administered at a dose selected from the group consisting of 200 mg, 400 mg, 500 mg, 600 mg, 800 mg and 1200 mg once every three weeks, wherein the subject having said GCT received no previous systemic treatment for GCT.
28. A method of treating an ovarian granulosa cell tumor (GCT) containing a FOXL2 C134W mutation, comprising administering to a human subject having said GCT the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose selected from the group consisting of 200 mg, 400 mg, 500 mg, 550 mg, 600 mg, 1000 mg, 1500 mg, and 1600 mg once every four weeks.
29. A method of embodiment 28, wherein the anti-TGF-β1/GARP complex antibody is administered at a dose of 500 mg.
30. A method of embodiment 28, wherein the anti-TGF-β1/GARP complex antibody is administered at a dose of 600 mg.
31. A method of embodiment 28, wherein the anti-TGF-β1/GARP complex antibody is administered at a dose of 1500 mg.
32. A method of treating an ovarian granulosa cell tumor (GCT) containing a FOXL2 C134W mutation in a population of human subjects, comprising administering to the human subjects having said GCT the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose ranging from about 200 mg to about 1500 mg once every two weeks, once every three weeks, or once every four weeks,
wherein the overall response rate (ORR) is greater than the standard of care, such as from 5% or greater, from about 10% or greater, from about 15% or greater, from 20% or greater, 30% or greater, or 40% or greater.
33. A TGF-β1/GARP complex antibody for use in combination with an anti-PD-1 antibody in the treatment of GCT, wherein the anti-TGF-β1/GARP complex antibody consists of two heavy chains each consisting of the amino acid sequence of SEQ ID NO:9 and two light chains each consisting of the amino acid sequence of SEQ ID NO:10, and wherein the anti-TGF-β1/GARP complex antibody is administered at a dose ranging from about 200 mg to about 1500 mg once every two weeks, once every three weeks or once every four weeks.

### 8.7. Head and Neck Squamous Cell Carcinoma Embodiments

1. A method of treating a head and neck squamous cell carcinoma, comprising administering to a human subject having said head and neck squamous cell carcinoma the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose ranging from about 200 mg to about 1600 mg once every two weeks, once every three weeks, or once every four weeks.
2. The method of embodiment 1, wherein the anti-TGF-β1/GARP complex antibody is administered once every two weeks at a dose ranging from about 200 mg to about 1500 mg.
3. The method of embodiment 1, wherein the anti-TGF-β1/GARP complex antibody is administered once every three weeks at a dose ranging from about 200 mg to about 1200 mg.
4. The method of embodiment 1, wherein the anti-TGF-β1/GARP complex antibody is administered once every four weeks at a dose ranging from about 200 mg to about 1600 mg.
5. The method of embodiment 1, wherein the anti-TGF-β1/GARP complex antibody is administered at a dose selected from the group consisting of 200 mg, 400 mg, 500 mg, 600 mg, 800 mg, 1000 mg, 1200 mg, 1500 mg, and 1600 mg.
6. The method of embodiment 2, wherein the anti-TGF-β1/GARP complex antibody is administered at a dose of 1500 mg.
7. The method of embodiment 3, wherein the anti-TGF-β1/GARP complex antibody is administered at a dose of 400 mg.
8. The method of embodiment 3, wherein the anti-TGF-β1/GARP complex antibody is administered at a dose of 600 mg.
9. The method of embodiment 3, wherein the anti-TGF-β1/GARP complex antibody is administered at a dose of 1200 mg.
10. The method of embodiment 4, wherein the anti-TGF-β1/GARP complex antibody is administered at a dose of 500 mg.
11. The method of embodiment 4, wherein the anti-TGF-β1/GARP complex antibody is administered at a dose of 600 mg.
12. The method of embodiment 4, wherein the anti-TGF-β1/GARP complex antibody is administered at a dose of 1500 mg.
13. The method of embodiment 1, wherein head and neck squamous cell carcinoma arose from the oral cavity, oropharynx, hypopharynx, or larynx.
14. The method of embodiment 1, wherein the subject having said head and neck squamous cell carcinoma received a prior treatment for first-line treatment for head and neck squamous cell carcinoma and experienced disease progression on the prior treatment.
15. The method of embodiment 14, wherein the prior line of treatment was a platinum-based regimen and an anti-PD-1 or anti-PD-L1 antibody administered in the recurrent or metastatic setting.
16. The method of embodiment 1, wherein the anti-TGF-β1/GARP complex antibody is administered intravenously prior to the intravenous administration of the anti-PD-1 antibody, and the anti-TGF-β1/GARP complex antibody and the anti-PD-1 antibody are administered on the same day as one another.
17. The method of embodiment 1, wherein the anti-PD-1 antibody is budigalimab.
18. The method of embodiment 2, wherein the anti-PD-1 antibody is budigalimab administered at a dose of 500 mg once every four weeks.
19. The method of embodiment 3, wherein the anti-PD-1 antibody is budigalimab, and the anti-PD-1 antibody is administered at a dose of 375 mg once every three weeks.
20. The method of embodiment 4, wherein the anti-PD-1 antibody is budigalimab administered at a dose of 500 mg once every four weeks.
21. The method of embodiment 1, wherein the administration of the anti-TGF-β1/GARP complex antibody and the anti-PD-1 antibody are administered intravenously.
22. The method of embodiment 1, wherein the subject having said head and neck squamous cell carcinoma received no previous treatment for head and neck squamous cell carcinoma.
23. A method of treating head and neck squamous cell carcinoma, comprising administering to a human subject having said head and neck squamous cell carcinoma the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose selected from the group consisting of 200 mg, 400 mg, 500 mg, 600 mg, 800 mg and 1200 mg once every three weeks, and the anti-PD-1 antibody is administered at a dose of 375 mg once every three weeks,
wherein the subject having said head and neck squamous cell carcinoma received a prior line of treatment for head and neck squamous cell carcinoma and experienced disease progression on the prior line of treatment.
24. A method of embodiment 23, wherein the anti-TGF-β1/GARP complex antibody is administered at a dose of 400 mg.
25. A method of embodiment 23, wherein the anti-TGF-β1/GARP complex antibody is administered at a dose of 600 mg.
26. A method of embodiment 23, wherein the anti-TGF-β1/GARP complex antibody is administered at a dose of 1200 mg.
27. A method of treating a head and neck squamous cell carcinoma, comprising administering to a human subject having said head and neck squamous cell carcinoma the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose selected from the group consisting of 200 mg, 400 mg, 500 mg, 550 mg, 600 mg, 1000 mg, 1500 mg, and 1600 mg once every four weeks, and the anti-PD-1 antibody is administered at a dose of 500 mg once every four weeks.
28. A method of embodiment 27, wherein the anti-TGF-β1/GARP complex antibody is administered at a dose of 500 mg.
29. A method of embodiment 27, wherein the anti-TGF-β1/GARP complex antibody is administered at a dose of 600 mg.
30. A method of embodiment 27, wherein the anti-TGF-β1/GARP complex antibody is administered at a dose of 1500 mg.
31. A method of treating a head and neck squamous cell carcinoma in a population of human subjects, comprising administering to the human subjects having said head and neck squamous cell carcinoma the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose ranging from about 200 mg to about 1500 mg once every two weeks, once every three weeks, or once every four weeks, wherein the overall response rate (ORR) is greater than the standard of care, such as from 5% or greater, from 10% or greater, from 15% or greater, from 20% or greater, 30% or greater, 40% or greater.
33. A TGF-β1/GARP complex antibody for use in combination with an anti-PD-1 antibody in the treatment of head and neck squamous cell carcinoma, wherein the anti-TGF-β1/GARP complex antibody consists of two heavy chains each consisting of the amino acid sequence of SEQ ID NO:9 and two light chains each consisting of the amino acid sequence of SEQ ID NO:10, and wherein the anti-TGF-β1/GARP complex antibody is administered at a dose ranging from about 200 mg to about 1600 mg once every two weeks, once every three weeks or once every four weeks.

### 8.8. Pancreatic Adenocarcinoma Embodiments

1. A method of treating a pancreatic adenocarcinoma, comprising administering to a human subject having said pancreatic adenocarcinoma the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose ranging from about 200 mg to about 1600 mg once every two weeks, once every three weeks, or once every four weeks.
2. The method of embodiment 1, wherein the anti-TGF-β1/GARP complex antibody is administered once every two weeks at a dose ranging from about 200 mg to about 1500 mg.
3. The method of embodiment 1, wherein the anti-TGF-β1/GARP complex antibody is administered once every three weeks at a dose ranging from about 200 mg to about 1200 mg.
4. The method of embodiment 1, wherein the anti-TGF-β1/GARP complex antibody is administered once every four weeks at a dose ranging from about 200 mg to about 1600 mg.
5. The method of embodiment 1, wherein the anti-TGF-β1/GARP complex antibody is administered at a dose selected from the group consisting of 200 mg, 400 mg, 500 mg, 600 mg, 800 mg, 1000 mg, 1200 mg, 1400 mg, and 1500 mg.
6. The method of embodiment 2, wherein the anti-TGF-β1/GARP complex antibody is administered at a dose of 1500 mg
7. The method of embodiment 3, wherein the anti-TGF-β1/GARP complex antibody is administered at a dose of 400 mg.
8. The method of embodiment 3, wherein the anti-TGF-β1/GARP complex antibody is administered at a dose of 600 mg.
9. The method of embodiment 3, wherein the anti-TGF-β1/GARP complex antibody is administered at a dose of 1200 mg.
10. The method of embodiment 4, wherein the anti-TGF-β1/GARP complex antibody is administered at a dose of 500 mg.
11. The method of embodiment 4, wherein the anti-TGF-β1/GARP complex antibody is administered at a dose of 600 mg.
12. The method of embodiment 4, wherein the anti-TGF-β1/GARP complex antibody is administered at a dose of 1500 mg.
13. The method of embodiment 1, wherein the subject having said pancreatic adenocarcinoma received no more than one prior line of treatment for pancreatic adenocarcinoma and experienced disease progression on the first-line treatment.
14. The method of embodiment 13, wherein the prior line of treatment was
a) gemcitabine monotherapy or in combination with other agents;
b) FOLFIRINOX or another regimen including both 5-fluorouracil and oxaliplatin; or
c) capecitabine monotherapy or in combination with other agents,
administered in the adjuvant, locally advanced, or metastatic setting, and the subject had no prior exposure to a PD-1 or PD-L1 antagonist.
15. The method of embodiment 14, wherein the prior line of treatment was in an adjuvant setting, and disease progression occurred within six months of completing adjuvant therapy.
16. The method of embodiment 1, wherein the anti-TGF-β1/GARP complex antibody and the anti-PD-1 antibody are administered on the same day as one another.
17. The method of embodiment 1, wherein the anti-PD-1 antibody is budigalimab.
18. The method of embodiment 2, wherein the anti-PD-1 antibody is budigalimab administered at a dose of 500 mg once every four weeks.
19. The method of embodiment 3, wherein the anti-PD-1 antibody is budigalimab administered at a dose of 375 mg once every three weeks.
20. The method of embodiment 4, wherein the anti-PD-1 antibody is budigalimab administered at a dose of 500 mg once every four weeks.
21. The method of embodiment 1, wherein the administration of the anti-TGF-β1/GARP complex antibody and the anti-PD-1 antibody are administered intravenously.
22. The method of embodiment 1, wherein the subject having said pancreatic adenocarcinoma received no previous treatment for pancreatic adenocarcinoma.
23. A method of treating pancreatic adenocarcinoma, comprising administering to a human subject having said pancreatic adenocarcinoma the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose selected from the group consisting of 400 mg, 500 mg, 600 mg, 800 mg and 1200 mg once every three weeks, and the anti-PD-1 antibody is administered at a dose of 375 mg once every three weeks,
wherein the subject having said pancreatic adenocarcinoma received a prior line of treatment for pancreatic adenocarcinoma and experienced disease progression on the prior line of treatment.
24. The method of embodiment 23, wherein the anti-TGF-β1/GARP complex antibody is administered at a dose of 400 mg.
25. The method of embodiment 23, wherein the anti-TGF-β1/GARP complex antibody is administered at a dose of 600 mg.
26. The method of embodiment 23, wherein the anti-TGF-β1/GARP complex antibody is administered at a dose of 1200 mg.
27. The method of treating a pancreatic adenocarcinoma, comprising administering to a human subject having said pancreatic adenocarcinoma a therapeutically effective amount of the combination of
a) an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose selected from the group consisting of 200 mg, 400 mg, 500 mg, 550 mg, 600 mg, 1000 mg and 1500 mg once every four weeks, and the anti-PD-1 antibody is administered at a dose of 500 mg once every four weeks.
28. The method of embodiment 27, wherein the anti-TGF-β1/GARP complex antibody is administered at a dose of 500 mg.
29. The method of embodiment 27, wherein the anti-TGF-β1/GARP complex antibody is administered at a dose of 600 mg.
30. The method of embodiment 27, wherein the anti-TGF-β1/GARP complex antibody is administered at a dose of 1500 mg.
31. A method of treating a pancreatic adenocarcinoma in a population of human subjects, comprising administering to the human subjects having said pancreatic adenocarcinoma the combination of
a) a therapeutically effective amount of an anti-PD-1 antibody and
b) an anti-TGF-β1/GARP complex antibody consisting of two heavy chains each consisting of the amino acid sequence of SEQ ID NO:9 and two light chains each consisting of the amino acid sequence of SEQ ID NO:10,
wherein the anti-TGF-β1/GARP complex antibody is administered at a dose ranging from about 200 mg to about 1500 mg once every two weeks, once every three weeks, or once every four weeks, and the anti-PD-1 antibody is administered at a dose of 375 mg once every three weeks or 500 mg once every four weeks, wherein the overall response rate (ORR) is greater than the standard of care, such as from 5% or greater, from 10% or greater, from 15% or greater, from 20% or greater, 30% or greater, 40% or greater.
32. A TGF-β1/GARP complex antibody for use in combination with an anti-PD-1 antibody in the treatment of pancreatic adenocarcinoma, wherein the anti-TGF-β1/GARP complex antibody consists of two heavy chains each consisting of the amino acid sequence of SEQ ID NO:9 and two light chains each consisting of the amino acid sequence of SEQ ID NO:10, and wherein the anti-TGF-β1/GARP complex antibody is administered at a dose ranging from about 200 mg to about 1600 mg once every two weeks, once every three weeks or once every four weeks.

We claim:

1. A method of treating CMS4 enriched microsatellite stable colorectal cancer, comprising administering to a human subject having said cancer a therapeutically effective amount of the combination of
a) a therapeutically effective amount of an anti-PD-1 antibody on a therapeutically effective schedule and
b) livmoniplimab
wherein the livmoniplimab is administered at a dose ranging from about 200 mg to about 1600 mg once every two weeks, once every three weeks or once every four weeks.

2. The method of claim 1, wherein the anti-PD-1 antibody is budigalimab, and wherein the budigalimab is administered at a dose of 250 mg once every two weeks, 375 mg once every three weeks, or 500 mg once every 4 weeks.

3. A method of treating microsatellite stable colorectal cancer comprising administering to a human subject having said cancer a therapeutically effective amount of the combination of
a) a therapeutically effective amount of an anti-PD-1 antibody on a therapeutically effective schedule and
b) livmoniplimab
wherein the livmoniplimab is administered at a dose ranging from about 200 mg to about 1600 mg once every two weeks, once every three weeks or once every four weeks.

4. The method of claim 3, wherein the anti-PD-1 antibody is budigalimab, and wherein the budigalimab is administered at a dose of 250 mg once every two weeks, 375 mg once every three weeks, or 500 mg once every 4 weeks.

5. A method of treating non-small cell lung cancer (NSCLC), comprising administering to a human subject having said NSCLC a therapeutically effective amount of the combination of
a) a therapeutically effective amount of an anti-PD-1 antibody on a therapeutically effective schedule and
b) livmoniplimab
wherein the livmoniplimab is administered at a dose ranging from about 200 mg to about 1600 mg once every two weeks, once every three weeks or once every four weeks.

6. The method of claim 5, further comprising administration of carboplatin and pemetrexed.

7. The method of claim 5 or claim 6, wherein the anti-PD-1 antibody is budigalimab, and wherein the budigalimab is administered at a dose of 250 mg once every two weeks, 375 mg once every three weeks, or 500 mg once every 4 weeks.

8. A method of treating relapsed/refractory non-small cell lung cancer (NSCLC), comprising administering to a human subject having said cancer a therapeutically effective amount of the combination of
a) a therapeutically effective amount of an anti-PD-1 antibody on a therapeutically effective schedule and
b) livmoniplimab
wherein the livmoniplimab is administered at a dose ranging from about 200 mg to about 1600 mg once every two weeks, once every three weeks or once every four weeks.

9. The method of claim 1, wherein the anti-PD-1 antibody is budigalimab, and wherein the budigalimab is administered at a dose of 250 mg once every two weeks, 375 mg once every three weeks, or 500 mg once every 4 weeks.

10. A method of treating pancreatic adenocarcinoma comprising administering to a human subject having said pancreatic adenocarcinoma a therapeutically effective amount of the combination of
a) a therapeutically effective amount of an anti-PD-1 antibody on a therapeutically effective schedule and
b) livmoniplimab
wherein the livmoniplimab is administered at a dose ranging from about 200 mg to about 1600 mg once every two weeks, once every three weeks or once every four weeks.

11. The method of claim 10, wherein the anti-PD-1 antibody is budigalimab, and wherein the budigalimab is administered at a dose of 250 mg once every two weeks, 375 mg once every three weeks, or 500 mg once every 4 weeks.

12. The method of claim 10 or claim 11, further comprising the administration of paclitaxel or nab-paclitaxel and gemcitabine.

13. A method of treating muscle invasive bladder cancer comprising administering to a human subject having said cancer the combination of
a) a therapeutically effective amount of an anti-PD-1 antibody on a therapeutically effective schedule and
b) livmoniplimab
wherein the livmoniplimab is administered at a dose ranging from about 200 mg to about 1600 mg once every two weeks, once every three weeks or once every four weeks.

14. The method of claim 13, wherein the anti-PD-1 antibody is budigalimab, and wherein the budigalimab is administered at a dose of 250 mg once every two weeks, 375 mg once every three weeks, or 500 mg once every 4 weeks.

15. A method of treating ovarian granulosa cell tumor (GCT) containing a FOXL2 C134W mutation comprising administering to a human subject having said tumor a
a) a therapeutically effective amount of an anti-PD-1 antibody on a therapeutically effective schedule and
b) livmoniplimab
wherein the livmoniplimab is administered at a dose ranging from about 200 mg to about 1600 mg once every two weeks, once every three weeks or once every four weeks.

16. The method of claim 15, wherein the anti-PD-1 antibody is budigalimab, and wherein the budigalimab is administered at a dose of 250 mg once every two weeks, 375 mg once every three weeks, or 500 mg once every 4 weeks.

17. A method of treating head and neck squamous cell carcinoma comprising administering to a human subject having said carcinoma a therapeutically effective amount of the combination of
a) a therapeutically effective amount of an anti-PD-1 antibody on a therapeutically effective schedule and
b) livmoniplimab
wherein the livmoniplimab is administered at a dose ranging from about 200 mg to about 1600 mg once every two weeks, once every three weeks or once every four weeks.

18. The method of claim 1, wherein the anti-PD-1 antibody is budigalimab, and wherein the budigalimab is administered at a dose of 250 mg once every two weeks, 375 mg once every three weeks, or 500 mg once every 4 weeks.

19. A method of treating hepatocellular carcinoma (HCC) comprising administering to a human subject having said carcinoma a therapeutically effective amount of the combination of
a) a therapeutically effective amount of an anti-PD-1 antibody on a therapeutically effective schedule and
b) livmoniplimab
wherein the livmoniplimab is administered at a dose ranging from about 200 mg to about 1600 mg once every two weeks, once every three weeks or once every four weeks.

20. The method of claim 18, wherein the anti-PD-1 antibody is budigalimab, and wherein the budigalimab is administered at a dose of 250 mg once every two weeks, 375 mg once every three weeks, or 500 mg once every 4 weeks.

21. The method of claim 19 or claim 20, wherein the subject had no previous lines of treatment for HCC

22. The method of claim 19 or claim 20, wherein the subject had one previous line of treatment for HCC.

23. The method of claim 19 or claim 20, wherein the subject had more than one previous line of treatment for HCC.

24. A method of treating urothelial cancer comprising administering to a human subject having said cancer a therapeutically effective amount of the combination of
a) a therapeutically effective amount of an anti-PD-1 antibody on a therapeutically effective schedule and
b) livmoniplimab
wherein the livmoniplimab is administered at a dose ranging from about 200 mg to about 1600 mg once every two weeks, once every three weeks or once every four weeks.

25. The method of claim 1, wherein the anti-PD-1 antibody is budigalimab, and wherein the budigalimab is administered at a dose of 250 mg once every two weeks, 375 mg once every three weeks, or 500 mg once every 4 weeks.

26. The method of any one of claims 1-25, wherein administration of the combination of livmoniplimab and the anti-PD-1 antibody is more efficacious than is the anti-PD-1 antibody when used as a monotherapy.

27. The method of any one of claims 1-25, wherein administration of the combination of livmoniplimab and the anti-PD-1 antibody has a higher overall response rate than does the anti-PD-1 antibody when used as a monotherapy.


SEQUENCE LISTING (22 sequences)

SEQ ID NO: 1 - protein, 5 aa, synthetic construct
SYYID

SEQ ID NO: 2 - protein, 17 aa, synthetic construct
RIDPEDAGTKYAQKFQG

SEQ ID NO: 3 - protein, 17 aa, synthetic construct
YEWETVVVGDLMYEYEY

SEQ ID NO: 4 - protein, 11 aa, synthetic construct
QASQSISSYLA

SEQ ID NO: 5 - protein, 7 aa, synthetic construct
GASRLKT

SEQ ID NO: 6 - protein, 9 aa, synthetic construct
QQYASVPVT

SEQ ID NO: 7 - protein, 126 aa, synthetic construct
QVQLVQPGAEVRKPGASVKVSCKASGYRFTSYYIDWVRQAPGQGLEWMGRIDPEDAGTKYAQKFQGRVTMTADTSTSTVYVELSSLRSEDTAVYYCARYEWETVVVGDLMYEYEYWGQGTLVTVSS

SEQ ID NO: 8 - protein, 107 aa, synthetic construct
DIQMTQSPSSLSASVGDRVTITCQASQSISSYLAWYQQKPGQAPKILIYGASRLKTGVPSRFSGSGSGTSFTLTISSLEPEDAATYYCQQYASVPVTFGQGTKVEIK

SEQ ID NO: 9 - protein, 452 aa, synthetic construct
QVQLVQPGAEVRKPGASVKVSCKASGYRFTSYYIDWVRQAPGQGLEWMGRIDPEDAGTKYAQKFQGRVTMTADTSTSTVYVELSSLRSEDTAVYYCARYEWETVVVGDLMYEYEYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 214 aa, synthetic construct
DIQMTQSPSSLSASVGDRVTITCQASQSISSYLAWYQQKPGQAPKILIYGASRLKTGVPSRFSGSGSGTSFTLTISSLEPEDAATYYCQQYASVPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 10 aa, synthetic construct
GYTFTHYGMN

SEQ ID NO: 12 - protein, 17 aa, synthetic construct
WVNTYTGEPTYADDFKG

SEQ ID NO: 13 - protein, 9 aa, synthetic construct
EGEGLGFGD

SEQ ID NO: 14 - protein, 16 aa, synthetic construct
RSSQSIVHSHGDTYLE

SEQ ID NO: 15 - protein, 7 aa, synthetic construct
KVSNRFS

SEQ ID NO: 16 - protein, 9 aa, synthetic construct
FQGSHIPVT

SEQ ID NO: 17 - protein, 118 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSS

SEQ ID NO: 18 - protein, 113 aa, synthetic construct
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKR

SEQ ID NO: 19 - protein, 448 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 219 aa, synthetic construct
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 447 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 453 aa, synthetic construct
QVQLVQPGAEVRKPGASVKVSCKASGYRFTSYYIDWVRQAPGQGLEWMGRIDPEDAGTKYAQKFQGRVTMTADTSTSTVYVELSSLRSEDTAVYYCARYEWETVVVGDLMYEYEYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK